(19) United States
(12) Patent Application Publication
(10) Pub. No.: US 2008/0009615 A1
(43) Pub. Date: Jan. 10, 2008

(54) Increasing starch extraction rate in cereals

(76) Inventors: Michael J. Giroux, Bozeman, MT (US); Craig F. Morris, Pullman, WA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/806,673

(22) Filed: Jun. 1, 2007

Related U.S. Application Data
(63) Continuation-in-part of application No. 10/447,541, filed on May 27, 2003.
Application No. 10/447,541 is a division of application No. 09/489,674, filed on Jan. 24, 2000, now Pat. No. 6,600,090.
Application No. 09/489,674 is a continuation-in-part of application No. 09/083,852, filed on May 22, 1998, now Pat. No. 6,596,930.

(51) Int. Cl. C07H 1/08; C08B 31/00

(52) U.S. Cl. 536/102; 536/128

(57) ABSTRACT

The present invention provides for control of seed grain hardness resulting in improved cereals for both agricultural feed and commercial food products for human consumption.

### CROSS REFERENCES TO RELATED APPLICATIONS

This application is a continuation-in-part of U.S. patent application Ser. No. 10/447,541, filed on May 27, 2003, which is a continuation of Ser. No. 09/489,674, filed on Jan. 24, 2000, issued as U.S. Pat. No. 6,600,900 on Jul. 29, 2003, which is in turn a continuation-in-part of U.S. patent application Ser. No. 09/083,852, filed on May 22, 1998, issued as U.S. Pat. No. 6,596,930 on Jul. 22, 2003, each of which is hereby incorporated by reference in their entireties, including any associated sequence listings.

### FEDERALLY FUNDED RESEARCH

This invention was made at least partly with government support under DE-FG3.6-02G012026 awarded by the Department of Energy; 2004-35301-14538 awarded by the U.S. Department of Agriculture; and Competitive Grant No. 2004-01141 awarded by the USDA-ARS National Research Initiative. The government may therefore have certain rights in this invention.

### FIELD OF THE INVENTION

This invention relates to grain hardness as a significant factor in determining the suitability of grains for grain extraction processes, particularly wet milling processes. More specifically, this invention relates to methods for producing transgenic grain plants which express puroindoline proteins, the progeny of such plants, and the plants and grains produced by such methods. The grain harvested from the transgenic grain plants of this invention demonstrate an increase in starch extractability and increased total recovery with highest yields seen in the transgenic line having intermediate grain texture.

### BACKGROUND

All publications and patent applications herein are incorporated by reference to the same extent as if each individual publication or patent application was specifically and individually indicated to be incorporated by reference.

Cereals, including wheat (Triticum aestivum L. em Thell.), are the most important food crops in the world. In addition to use in feed for livestock, the grain from cereals are milled into flour in almost every culture. Wheat flour is found extensively throughout the world but particularly in Europe and North America, rice flour is used extensively in Asia, sorghum flour in Africa, and corn flour (or meal) in the Americas.

The grain of cereal plants varies between species and within species of cereal plants. Grain texture refers to the texture of the kernel (caryopsis), that is, whether endosperm is physically hard or soft. Typically, rice, sorghum, barley and maize are hard textured grains while oat, rye and triticale are soft. Nearly all of the world production and trade in wheat (approximately 550 and 100 mmt annually, respectively) is identified as being either soft or hard. Generally speaking, hard wheat is used for bread whereas soft wheat is used for cookies, cakes and pastries (Morris & Rose, Cereal Grain Quality, Chapman & Hall, New York, N.Y., pp. 3-54 (1996)). The very hard durum wheat (T. turgidum) is generally used in pasta.

In addition to differences in taste and water absorption, grain texture dictates milling techniques. Typically, the harder the grain, the more energy is required for milling, the greater the starch damage during the milling process, and the larger the milled particle size.

A 15 kDa marker protein for grain softness, termed friabilin, is present on the surface of water-washed starch from soft wheats in high amounts, on hard wheat starch in small amounts, and absent on durum wheat starch (Greenwell & Schofield, Cereal Chem. 63:379-380 (1986)). N-terminal sequence analysis of friabilin indicates a mixture of two or more discrete polypeptides (Morris, et al., J. Cereal Sci. 21:167-174 (1994); and Jolly, et al., Theor. Appl. Genet. 86:589-597 (1993)). The two major component polypeptides have been found to be identical to the two lipid binding proteins termed puroindolines (Gautier, et al., Plant Molec. Biol. 25:43-57 (1994)), puroindoline A (referred to as either ‘puro A’ or ‘PINA’) and puroindoline B (referred to as either ‘puro B’ or ‘PINB’), respectively. The transcripts of puro A and puro B, are controlled by chromosome 5D (Giroux & Morris, Theor. Appl. Genet. 95:857-864 (1997)).

Puro A and puro B are unique among plant proteins because of their tryptophan-rich, hydrophobic domains, which have affinity for binding lipids (Blochet, et al., Gluten Proteins 1990, Bushuk & Tkachuk (eds), American Association of Cereal Chemists, St. Paul, Minn., pp. 314-325 (1991); and Wilde, et al., Agric. Res. 20:971 (1993)). The association of friabilin (puro A and puro B) with the surface of the starch granule is apparently mediated by polar lipids. In fact, the occurrence of membrane structural lipids, glyco- and phospho-lipids, with the surface of water washed starch follows that of friabilin (Greenblatt, et al., Cereal Chem. 72:172-176 (1995)): high amounts are present on soft wheat starch, low amounts on hard wheat starch, and none on durum.

There exists a need to modify the texture of grain in cereal plants with more certainty than is available by hybrid crossing. With hybrid crossing, there is the possibility that the parent plants will be reproductively incompatible. There also is the very real possibility that large amounts of water, fertilizer and acreage will be necessary to produce one hybrid plant. By creating transgenic plants with nucleic acid sequences that alter the texture of grain, these problems can be averted.

Cereal wet milling involves separation of a cereal seed into its useful components. The main products of wet milling are starch and protein. The recovery of starch and the purity of the starch recovered are important economically to those operating wet milling plants and to seed companies selling grain.

Maize (Zea mays L.), wheat (Triticum aestivum L.), rice (Oryza sativa) and potato Solanum tuberosum) are the four major sources of starch in the world (Sayaslan 2006). Wet milling is an important use of maize grain produced in the USA (Zehr et al 1996; Parris et al 2006). The development of high wet milling starch yield maize hybrids is very important in sweeteners and ethanol production in USA (Zehr et al 1996; Parris et al 2006). However, in Europe, wheat is becoming increasingly important as a starch source material (Bergthaller w. et al 2004). From the estimated 593.1 million metric tons (mmt) of annual wheat production in the world (USDA, WASDE-444 2007), 67% is used as food, 20% as feed and 7% as seed (Sayaslan et al 2006). The remaining 6% of production is used for industrial purposes, which includes wet milling to produce starch and vital gluten (Sayaslan et al 2006). World wheat starch production was ca. 4.1 million tons in 2000 (LMC International LTD, 2002), originating from ca. 8 million tons of wheat (Van Der Borght et al 2005).

The two major classes of hexaploid wheat (hard and soft wheat) have different milling and end use product characteristics (reviewed in Morris and Rose; 1996) mainly owing to allelic variation at the Hardness (Ha) locus located on the short arm of chromosome 5D (Mattern et al 1973; Law et al 1978). Mutations in the closely linked genes puroindolines a or b which comprise the Ha locus, have been found in all hard-textured wheat examined (Giroux and Morris 1997; 1998; Lillemo and Morris 2000; Morris et al 2001b; Cane et al 2004; Chen et al 2006). From a biochemical point of view, wheat grain hardness is most likely determined by the degree of adhesion between starch granules and protein matrix. The 15 kDa protein complex friabilin (Greenwell and Schofield, 1986; Morris et al 1994) consists of the structurally similar proteins puroindoline a and b, (PINA and PINB, respectively) expressed by the Ha locus (Giroux and Morris 1997, 1998). Friabilin may functions as a nonstick agent that decreases grain hardness via reduction of the close adherence between starch granules and wheat storage proteins (Anjum and Walker 1991). PINA and PINB are cysteine rich proteins which are unique among plant proteins in having a hydrophobic tryptophan rich domain (Blochet et al 1993). The tryptophan-rich domain is hypothesized to confer an affinity for lipids (Marion et al 1994; Kooijman et al 1997) and may result in PINA and PINB being localized to the glyco- and phospholipid rich surface of the amyloplast membrane (Giroux et al 2000). These protein-lipid interactions may be disrupted in hard wheats leading to hard endosperm texture.

Evidence that PINs affect hardness via binding to polar lipids came first in a report by Morrison et al (1989) who reported that grain hardness is negatively correlated with free polar lipids content. Similarly, a survey of several soft and hard wheat varieties demonstrated that higher amounts of glyco- and phospholipids were present on soft wheat starch than hard similar to friabilin levels in which soft wheat starch is friabilin rich and hard wheat starch is friabilin deficient (Greenblatt et al 1995). Defined genetic stocks, that differ in Ha function (Ha, soft vs. ha, hard) have been valuable not only in studying the biochemical and genetic basis of wheat grain hardness (Giroux and Morris 1997; 1998), but also provided material to study the effect of this gene on flour processing (Martin et al 2001; Morris and Allen 2001; Morris et al 2001a; Greffeuille v. et al 2006). In addition to natural genetic stocks, transgenic Pin addition lines have been successfully used both to prove the function of the Ha locus (Beecher et al 2002; Martin et al 2006) and to study the impact of this locus on flour processing and different end-use qualities (Hogg et al 2005; Swan et al 2006a; Martin et al 2007).

Both small and large scale investigations have suggested that wheat varieties with softer endosperm texture are not only advantageous in dry milling but also in the wet milling process (Bergthaller et al 2004; Czuchajowska and Pomeranz 1993). Small scale methods to simulate industrial scale wet milling have been used on wheat flour (Czuchajowska and Pomeranz 1993; Sayaslan et al 2006) or maize intact kernels (Vignaux et al 2006). In order to test for a linkage between grain hardness and Pin expression with starch extractability two independent groups of soft/hard isogenic lines were used in small scale laboratory wet milling tests. These were near isogenic lines (NILs) for the Ha locus in two genetic backgrounds, and transgenic isolines in hard red spring wheat ‘Hi-Line’ over expressing Pina, Pinb or both Pina and Pinb. The soft/hard isolines used allow comparison of relatively small (non-transgenic) and large (transgenic) variation in PIN expression level and grain hardness.

### SUMMARY OF THE INVENTION

This invention provides for the identification of puroindoline A and puroindoline B as the major components of grain softness in wheat (Triticum aestivum). This invention also provides for methods of introducing puroindoline genes and puroindoline homologs into wheat and other cereal plants to modify grain texture.

This invention provides plant cells, plant tissues and plants transgenic for nucleic acids encoding puroindolines.

This invention further provides plant cells, plant tissues and plants transgenic for nucleic acids which hybridize under high stringency conditions with nucleic acids encoding puroindolines.

This invention also provides plant cells, plant tissues and plants transgenic for nucleic acids encoding fragments of puroindolines wherein the fragments retain at least one biological activity of the puroindolines.

This invention further provides plant cells, plant tissues or plants transgenic for recombinant DNA sequences encoding either or both of puroindoline A and puroindoline B.

This invention further provides isolated nucleic acid molecules comprising nucleic acids operatively linked to constitutive or inducible promoters in a manner effective for expression of the nucleic acids, wherein the nucleic acids are selected from the group consisting of nucleic acids encoding one or more puroindolines, nucleic acids which hybridizes under high stringency conditions to the nucleic acids encoding puroindolines, and nucleic acids encoding fragments of a puroindoline wherein the fragments retain at least one biological activity of a puroindoline. This invention also provides methods of producing transformed plant cells, plant tissues or plants by transforming the plant cells, plant tissues or plants with such isolated nucleic acid molecules. This invention further provides methods of crossing the transformed plants to different plants, harvesting the resultant seeds, and planting and growing the harvested seeds.

In one embodiment, a method of producing a transgenic plant with softer textured grain from at least one parent plant with hard textured grain is provided. The method comprises the steps of introducing a nucleic acid sequence which hybridizes under stringent conditions to a nucleic acid sequence selected from the group consisting of SEQ ID NO:1 and SEQ ID NO:3 and operably encodes a puroindoline protein into a cell from the parent plant, and generating a plant from the cell containing the nucleic acid sequence. In another embodiment, the plant is selected from the group consisting of durum wheat, sorghum, rice, barley and maize. In still another embodiment, the plant is maize. In another embodiment, the introduction of the nucleic acid is mediated by Agrobacterium infection. Also in this embodiment, it is preferred that the puroindoline protein is selected from the group consisting of puroindoline A and puroindoline B.

In still another embodiment, a transgenic plant with soft textured grain is provided. The plant is derived from at least one parent plant which has hard textured grain. The plant comprises a nucleic acid sequence which operably encodes a puroindoline protein, wherein the nucleic acid sequence hybridizes under stringent conditions to a nucleic acid sequence selected from the group consisting of SEQ ID NO:1 and SEQ ID NO:3. In one embodiment, the plant is selected from the group consisting of durum wheat, sorghum, rice, barley and maize. In another embodiment, the plant is maize.

In one embodiment, the puroindoline nucleic acid sequence is introduced into the plant by transformation and the puroindoline protein is selected from the group consisting of puroindoline A and puroindoline B. In some embodiments, transformation is by Agrobacterium infection.

In yet another embodiment, this invention provides for a transgenic plant with hard grain derived from at least one parent having soft grain. The plant comprises a nucleic acid sequence which hybridizes under stringent conditions to a nucleic acid sequence selected from the group consisting of SEQ ID NO:5.

Expression of proteins that control grain hardness has been demonstrated by the present inventors. Controlling grain hardness using the compositions and methods of the present invention provides enhanced grain production, storage, digestibility, and palatability of grains such as barley and corn used as animal feeds, thereby providing improved animal weight gain and overall animal health. The better weight gain in animals results from delayed starch digestibility and improved palatability of the feed grains produced as a result of the present invention. The compositions and methods of the present invention help reduce grain spoilage and result in more efficient grain milling. Controlling grain hardness also provides benefits in human cereal food products through more efficient milling or improvements such as finer textured flours, advancements in barley malting, and enhanced starch extractability from grains such as corn.

The present invention provides plant compositions and methods to increase the ease with which starch and protein separate during wet milling by coating starch with the starch granule membrane puroindoline proteins to prevent tight adhesion between starch granules and storage proteins. We measured starch extraction on wheats that transgenically vary in puroindoline expression level. Our results demonstrate that increased puroindolines on starch increases both the amount and the purity of starch recovered after wet milling.

Advantages of the high extractable starch (“HES”) corn inbred and hybrid lines of the present invention include (1) being better suited for wet milling; (2) higher yields of starch than presently available HES lines and (3) the HES character utilized in the lines of the present invention are inherited as a single dominant gene.

### BRIEF DESCRIPTION OF THE SEQUENCES

SEQ ID NO:1 is the cDNA sequence of puroindoline A.

SEQ ID NO:2 shows the amino acid sequence encoded by SEQ ID NO:1.

SEQ ID NO:3 is the cDNA sequence of puroindoline B.

SEQ ID NO:4 shows the amino acid sequence encoded by SEQ ID NO:3.

SEQ ID NO:5 is the cDNA sequence of serine substituted puroindoline B.

SEQ ID NO:6 shows the amino acid sequence encoded by SEQ ID NO:5.

SEQ ID NO:7 is a sense strand primer for puroindoline A.

SEQ ID NO:8 is an antisense strand primer for puroindoline A.

SEQ ID NO:9 is a sense strand primer for puroindoline B.

SEQ ID NO:10 is an antisense strand for puroindoline B.

SEQ ID NO:11 is an antisense strand for serine substituted puroindoline B.

SEQ ID NO:12 is a GSP1 sense strand.

SEQ ID NO:13 is a GSP1 antisense strand.

### DETAILED DESCRIPTION OF THE INVENTION

This invention relates to plant puroindoline genes, in particular, wheat puroindoline A and puroindoline B genes. Nucleic acid sequences from puroindoline genes can be used to modify the texture of grain in both transgenic and progeny cereal plants. The puroindoline genes of this invention can be expressed in cereal species commonly used for production of flour, food stuffs and/or feed, e.g., wheat, rye, oats, maize and the like. By adding puroindoline genes to the genome of a cereal plant, the grain of the plant becomes softer textured than the grain of its parent. By blocking expression of puroindoline genes, the grain becomes harder than the grain of a cereal plant's parent. In addition, because of the quantitative nature of grain texture, introducing mutant forms of the puroindoline genes effects modifications in grain texture of cereal plants.

Generally, the nomenclature and the laboratory procedures in plant maintenance and breeding as well as recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook, et al., Molecular Cloning—A Laboratory Manual, Cold Spring Harbor Laboratory, Cold Spring Harbor, N.Y., (1989) and Dodds & Roberts, Experiments in Plant Tissue Culture, 3rd Ed., Cambridge University Press (1995).

I. Definitions

Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular biology (2d ed. 1994); The Cambridge Dictionary of Science and Technology (Walker ed., 1988); The Glossary of Genetics, 5th Ed., Rieger, R., et al. (eds.), Springer Verlag (1991); and Hale & Marham, The Harper Collins Dictionary of biology (1991). As used herein, the following terms have the meanings ascribed to them unless specified otherwise.

As used herein, the term “allele” means any of several alternative forms of a gene.

The term “cell” can refer to any cell from a plant, including but not limited to, somatic cells, gametes or embryos. “Embryo” refers to a sporophytic plant before the start of germination. Embryos can be formed by fertilization of gametes by sexual crossing or by selfing. A “sexual cross” is pollination of one plant by another. “Selfing” is the production of seed by self-pollination, i.e., pollen and ovule are from the same plant. The term “backcrossing” refers to crossing a F1 hybrid plant to one of its parents. Typically, backcrossing is used to transfer genes which confer a simply inherited, highly heritable trait into an inbred line. The inbred line is termed the recurrent parent. The source of the desired trait is the donor parent. After the donor and the recurrent parents have been sexually crossed, F1 hybrid plants which possess the desired trait of the donor parent are selected and repeatedly crossed (i.e., backcrossed) to the recurrent parent or inbred line.

Embryos can also be formed by “embryo somatogenesis” and “cloning.” Somatic embryogenesis is the direct or indirect production of embryos from cells, tissues and organs of plants. Indirect somatic embryogenesis is characterized by growth of a callus and the formation of embryos on the surface of the callus. Direct somatic embryogenesis is the formation of an asexual embryo from a single cell or group of cells on an explant tissue without an intervening callus phase. Because abnormal plants tend to be derived from a callus, direct somatic embryogenesis is preferred.

As used herein, the term “crop plant” means any plant grown for any commercial purpose, including, but not limited to the following purposes: seed production, hay production, ornamental use, fruit production, berry production, vegetable production, oil production, protein production, forage production, silage, animal grazing, golf courses, lawns, flower production, landscaping, erosion control, green manure, improving soil tilth/health, producing pharmaceutical products/drugs, producing food additives, smoking products, pulp production and wood production. Particular crop plants of interest to the present invention include, but are not limited to, wheat, rice, maize, barley, rye, sugar beets, potatoes, sweet potatoes, soybeans, cotton, tomatoes, canola and tobacco.

As used herein, the term “cross pollination” or “cross-breeding” means the pollen of one flower on one plant is applied (artificially or naturally) to the ovule (stigma) of a flower on another plant.

As used herein, the term “cultivar” means a variety, strain or race of plant which has been produced by horticultural or agronomic techniques and is not normally found in wild populations.

As used herein, the term “damage” is synonymous with “plant damage” and means any injury to a plant caused by any pest. Examples of plant damage include, but are not limited to, reduced shoot growth, reduced root growth, root pruning, necrotic leaf spots, lodging, wilting, stunting, chlorosis, broken tops, reduced branching, reduced flowering, flower abortion, ovule abortion, pollen abortion, yellowing of the leaf, lesions, internal stem discoloration, decayed roots, discolored stems, stalk tunneling, insect feeding, defoliation, reduced vigor, reduction in seed quality or viability and dead and dying plants.

As used herein, the terms “Dicotyledoneae”, “dicotyledonous”, “dicotyledon” or “dicot” are synonymous and mean any of various flowering plants having two embryonic seed leaves or cotyledons that usually appear at germination. Examples include, but are not limited to, tobacco, soybeans, potato, sweet potato, radish, cabbage, rape and apple trees.

As used herein, the term “disease” is synonymous with “plant disease” and means an infection by a pathogen.

The phrase “grain texture” refers to the main basis of classification of wheat grown for market.

The common term, “grain” is the endosperm present in the ovules of a plant. In wheat, texture of the grain or endosperm is distinguished by expression of the Hardness gene. However, all cereal grains can be classified on the basis of grain texture. In Sorghum and maize, softer endosperm results from mutations in genes such as opaque-2 and floury-2. However, expression of these mutant genes is recessive and leads to other, deleterious phenotypes such as greater susceptibility to mechanical and insect damage.

“Softer textured grain” refers to grain produced by a progeny or transgenic plant that is 10 units less than the grain produced by at least one of the plant's parent as measured by the Perten SKCS 4100 (Perten Instruments, Reno, Nev.); by near-infra red reflectance spectroscopy (NIR) as described in Method 39-70 (Approved Methods of the American Association of Cereal Chemists, 9th Ed., American Association of Cereal Chemists, St. Paul, Minn. (1995); or by equivalent technology. In a more preferred embodiment, the grain of the progeny or transgenic plant is at least 20 units lower than the grain of at least one of the parents. In a most preferred embodiment, the grain of the progeny or transgenic plant is at least 40 units lower than the grain of the parent. However, because grain hardness is a quantitative trait, one of skill will realize that hardness of grain is determined in part by the environment in which the progeny or transgenic plant and parent plants are grown.

“Harder textured grain” refers to grain produced by a progeny plant that is at least 10 units greater than the grain produced by at least one of the progeny plant's parent as measured by the techniques described above. In a more preferred embodiment, the grain of the progeny or transgenic plant is at least 20 units greater than the grain of at least one of the parents. In a most preferred embodiment, the grain of the progeny or transgenic plant is at least 40 units greater than the grain of the parent.

As used herein, the term “genotype” means the genetic makeup of an individual cell, cell culture, plant, or group of plants.

As used herein, the term “heterozygote” means a diploid or polyploid individual cell or plant having different alleles (forms of a given gene) at least at one locus.

As used herein, the term “heterozygous” means the presence of different alleles (forms of a given gene) at a particular gene locus.

As used herein, the term “homozygote” means an individual cell or plant having the same alleles at one or more loci.

As used herein, the term “homozygous” means the presence of identical alleles at one or more loci in homologous chromosomal segments.

As used herein, the term “hybrid” means any individual plant resulting from a cross between parents that differ in one or more genes.

As used herein, the term “inbred” or “inbred line” means a relatively true-breeding strain.

As used herein, a nucleic acid molecule is said to be “isolated” when the nucleic acid molecule is substantially separated from contaminant nucleic acid encoding other polypeptides from the source of nucleic acid.

As used herein, the term “line”, when directed to a type of plant, means self- or cross-fertilizing plants and single-line facultative apomicts, having largely the same genetic background, that are similar in essential and distinctive characteristics.

As used herein, the term “locus” (plural: “loci”) means any site that has been defined genetically. A locus may be a gene, or part of a gene, or a DNA sequence that has some regulatory role, and may be occupied by different sequences.

As used herein, the term “mass selection” means a form of selection in which individual plants are selected and the next generation propagated from the aggregate of their seeds.

As used herein, the terms “Monocotyledonieae”, “monocotyledonous”, “monnocotyledon” or “monocot” are synonymous and mean any of various flowering plants having a single cotyledon in the seed. Examples of monocots include, but are not limited to, rice, wheat, barley, maize and lilies.

As used herein, the term “Northern Blot” refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook et al., Molecular Cloning: A Laboratory Manual, 2nd edition, Cold Spring Harbor Laboratory Press, 1985).

As used herein, the term “open pollination” means a plant population that is freely exposed to some gene flow, as opposed to a closed one in which there is an effective barrier to gene flow.

As used herein, the terms “open-pollinated population” or “open-pollinated variety” mean plants normally capable of at least some cross-fertilization, selected to a standard, that may show variation but that also have one or more genotypic or phenotypic characteristics by which the population or the variety can be differentiated from others. A hybrid which has no barriers to cross-pollination is an open-pollinated population or an open-pollinated variety.

As used herein, the term “ovule” means the female gametophyte, whereas the term “pollen” means the male gametophyte.

As used herein, the term “pathogen” means a disease-producing agent, especially a microorganism. Examples of pathogens include, but are not limited to, bacteria, fungi, viruses and nematodes.

As used herein, the term “phenotype” means the observable characters of an individual cell, cell culture, plant, or group of plants which results from the interaction between that individual's genetic makeup (i.e., genotype) and the environment.

As used herein, the term “progeny” means the descendants of a particular plant (self-cross) or pair of plants (crossed or backcrossed). The descendants can be of the F1, the F2, or any subsequent generation. Typically, the parents are the pollen donor and the ovule donor which are crossed to make the progeny plant of this invention. Parents also refer to F1 parents of a hybrid plants of this invention (the F2 plants). Finally, parents refer to a recurrent parent which is backcrossed to hybrid plants of this invention to produce another hybrid plant of this invention.

As used herein, the term “Polymerase Chain Reaction” is synonymous with “PCR” and refers to techniques in which cycles of denaturation, annealing with primer, and extension with DNA polymerase, are used to amplify the number of copies of a target DNA sequence.

As used herein, the term “resistance” means a host plant's more or less total capacity to fight a pest, wherein the resistance is usually due to a gene-for-gene resistance.

As used herein, the term “rice” means any Oryza species, including, but not limited to, O. sativa, O. glaberrima, O. perennis, O. nivara, and O. breviligulata. Thus, as used herein, the term “rice” means any type of rice including, but is not limited to, any cultivated rice, any wild rice, any rice species, any intra- and inter-species rice crosses, all rice varieties, all rice genotypes and all rice cultivars.

As used herein, the term “self pollinated” or “self-pollination” means the pollen of one flower on one plant is applied (artificially or naturally) to the ovule (stigma) of the same or a different flower on the same plant.

As used herein, the term “synthetic” means a set of progenies derived by intercrossing a specific set of clones or seed-propagated lines. A synthetic may contain mixtures of seed resulting from cross-, self-, and sib-fertilization.

As used herein, the term “tolerance” means a host plant's partial capacity to fight a pest, wherein the tolerance is not necessarily due to a gene-for-gene resistance.

As used herein, the term “tomato” means any Lycopersicon species, including, but not limited to, L. cheesmanii Riley, L. chilense Dun., L. esculentum f. pyriforme (Dun.) C. H. Muller, L. esculentum Mill., L. esculmentum var. cerasiforme (Dun.) A. Gray, L. hirsutum Humb. & Bonpl., L. peruvianum (L.) Mill., and L. pimpinellifolium (L.) Mill. Thus, as used herein, the term “tomato” means any type of tomato including, but is not limited to, any cultivated tomato, any wild tomato, any tomato species, any intra- and inter-species tomato crosses, all tomato varieties, all tomato genotypes and all tomato cultivars. Cultivated tomatoes include, but are not limited to, pear tomatoes, Italian tomatoes, cherry tomatoes, canning tomatoes, sauce tomatoes and beef tomatoes.

As used herein, the term “transformation” means the transfer of nucleic acid (i.e., a nucleotide polymer) into a cell. As used herein, the term “genetic transformation” means the transfer and incorporation of DNA, especially recombinant DNA, into a cell.

As used herein, the term “transgenic” means cells, cell cultures, plants, and progeny of plants which have received a foreign or modified gene by one of the various methods of transformation, wherein the foreign or modified gene is from the same or different species than the species of the plant receiving the foreign or modified gene. As used herein, the terms “transgenic plant” and “transformed plant” are synonymous, as are the terms “transgenic line” and “transformed line”. As used herein, the phrases “corresponding non-transgenic plant” and “corresponding non-transgenic line” refer to the cells, cell cultures, plants and progeny of plants which did not receive the foreign or modified gene which the “transgenic” cells, cell cultures, plants and progeny of plants which did receive the foreign or modified gene.

As used herein, the term “variety” means a subdivision of a species, consisting of a group of individuals within the species which are distinct in form or function from other similar arrays of individuals.

As used herein, the term “wheat” means any Triticum species, including, but not limited to, T. aestivum, T. monococcum, T. tauschii and T. turgidum. Thus, as used herein, the term “wheat” means any type of wheat including, but is not limited to, any cultivated wheat, any wild wheat, any wheat species, any intra- and inter-species wheat crosses, all wheat varieties, all wheat genotypes and all wheat cultivars. Cultivated wheats include, but are not limited to, einkom, durum and common wheats.

As used herein, “wet milling” means the separation of cereal seed fractions under aqueous conditions. For example, wet milling generally involves steeping grain or flour in water and the resulting separation of the grain or flour into different components. Either whole grain, pieces of whole grain, flour or any combination of these things can be used in the wet milling processes of the present invention.

The common names of plants used throughout this disclosure refer to varieties of plants of the following genera:

| Common Name | Genera |
|---|---|
| Wheat (soft, hard and durum varieties) | Triticum |
| Sorghum | Sorghum |
| Rice | Oryza |
| Barley | Hordeum |
| Maize or corn | Zea |
| Rye | Secale |
| Triticale | Triticale |
| Oat | Avena |

II. Nucleic Acids Encoding Puroindolines

Gautier et al. (1994, Plant Mol. Bio. 25:43-57) isolated and sequenced cDNA clones encoding the two puroindolines from a mid-maturation seed cDNA library (see FIGS. 1, 2 and 3 of the article) (see also, GenBank Accession Numbers X69912 (SEQ ID NO. 3), X69913 and X69914 (SEQ ID NO. 4) for the nucleotide sequences; GenBank Accession Numbers S46514 (SEQ ID NO. 4), S46515, CAA49538, and CAA49539 (SEQ ID NO. 2) for the amino acid sequences). The Gautier et al. article and the associated sequences, including FIGS. 1, 2 and 3 of the article and the associated GenBank accessions, are specifically incorporated by reference herein in their entirety.

As used herein, puroindoline genes include the specifically identified and characterized variants herein described as well as allelic variants, conservative substitution variants and homologues that can be isolated/generated and characterized without undue experimentation following methods well known to one skilled in the art.

Homology or identity at the amino acid or nucleotide level is determined by BLAST (Basic Local Alignment Search Tool) analysis using the algorithm employed by the programs blastp, blastn, blastx, tblastn and tblastx (Karlin et al., 1990, Proc. Natl. Acad. Sci. USA 87, 2264-2268 and Altschul, 1993, J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., 1994, Nature Genetics 6, 119-129 which is fully incorporated by reference). The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., 1992, Proc. Natl. Acad. Sci. USA 89, 10915-10919, fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

The terms “puroindoline genes”, “pinA genes”, or “pinB genes” include all naturally occurring allelic variants of the puroindoline genes exemplified herein.

The puroindoline nucleic acid molecules or fragment thereof utilized in the present invention may also be synthesized using methods known in the art. It is also possible to produce the molecule by genetic engineering techniques, by constructing DNA using any accepted technique, cloning the DNA in an expression vehicle and transfecting the vehicle into a cell which will express the puroindoline proteins. See, for example, the methods set forth in Sambrook et al., Molecular Cloning: A Laboratory Manual, 2nd edition, Cold Spring Harbor Laboratory Press, 1985.

The phrase “puroindoline protein” refers to a class of proteins, including but not limited to, “puroindoline A” or “puro A” or “PINA” and “puroindoline B” or “puro B” or “PINB”. Puro A and puro B have tryptophan-rich hydrophobic domains which have affinity for binding lipids, referred to herein as “the lipid-binding domains” (Blocket et al., 1991, Gluten Proteins 1990, Bushak & Tkachuk (eds.), American Association of Cereal Chemists, St. Paul, Minn.; Wilde et al., 1993, Agric. Res. 20:971)). It is understood that all polynucleotides encoding all or a portion of the puroindoline proteins used in the present invention are also included herein, as long as they encode a polypeptide with one or more of the functional activities of the puroindoline proteins as set forth herein. Thus, any polynucleotide fragment having the activities of the puroindolines discussed herein are encompassed by the present invention.

Polynucleotide sequences of the invention include DNA, cDNA, synthetic DNA and RNA sequences which encode puroindoline proteins. Such polynucleotides also include naturally occurring, synthetic and intentionally manipulated polynucleotides. For example, such polynucleotide sequences may comprise genomic DNA which may or may not include naturally occurring introns. Moreover, such genomic DNA may be obtained in association with promoter regions or poly A sequences. As another example, portions of the mRNA sequence may be altered due to alternate RNA splicing patterns or the use of alternate promoters for RNA transcription. As yet another example, puroindoline polynucleotides may be subjected to site-directed mutagenesis.

The polynucleotides of the invention further include sequences that are degenerate as a result of the genetic code. The genetic code is said to be degenerate because more than one nucleotide triplet codes for the same amino acid. There are 20 natural amino acids, most of which are specified by more than one codon. It will be appreciated by those skilled in the art that as a result of the degeneracy of the genetic code, a multitude of nucleotide sequences, some bearing minimal nucleotide sequence homology to the nucleotide sequence of pinA and pinB may be utilized in the present invention. Therefore, all degenerate nucleotide sequences are included in the invention as long as the amino acid sequence of the puroindoline polypeptides encoded by the nucleotide sequence are functionally unchanged or substantially similar in function. The invention specifically contemplated each and every possible variation of peptide or nucleotide sequence that could be made by selecting combinations based on the possible amino acid and codon choices made in accordance with the standard triplet genetic code as applied to the puroindoline sequences of the invention, as exemplified by pinA and pinB, and all such variations are to be considered specifically disclosed herein.

Also included in the invention are fragments (portions, segments) of the sequences disclosed herein which selectively hybridize to pinA and pinB. Selective hybridization as used herein refers to hybridization under stringent conditions (See, for example, the techniques in Maniatis et al., Molecular Cloning: A Laboratory Manual, Cold Spring Harbor Laboratory Press, 1989), which distinguishes related from unrelated nucleotide sequences. The active fragments of the invention, which are complementary to mRNA and the coding strand of DNA, are usually at least about 15 nucleotides, more usually at least 20 nucleotides, preferably 30 nucleotides and more preferably may be 50 nucleotides or more.

“Stringent conditions” are those that (1) employ low ionic strength and high temperature for washing, for example, 0.5 M sodium phosphate buffer pH 7.2, 1 mM EDTA pH 8.0 in 7% SDS at either 65 degrees C. or 55 degrees C., or (2) employ during hybridization a denaturing agent such as formamide, for example, 50% (vol/vol) formamide with 0.1% bovine serum albumin, 0.1% Ficoll, 0.1% polyvinylpyrrolidone, 0.05 M sodium phosphate buffer at pH 6.5 with 0.75 M NaCl, 0.075 M sodium citrate at 42 degrees C. Another example is use of 50% formamide, 5.times.SSC (0.75 M NaCl, 0.075 M sodium citrate), 50 mM sodium phosphate (pH 6.8), 0.1% sodium pyrophosphate, 5.times.Denhardt's solution, sonicated salmon sperm DNA (50 micro g/ml), 0.1% SDS, and 10% dextran sulfate at 55 degrees C., with washes at 55 degrees C. in 0.2.times.SSC and 0.1% SDS. A skilled artisan can readily determine and vary the stringency conditions appropriately to obtain a clear and detectable hybridization signal. Preferred molecules are those that hybridize under the above conditions to the complements of pinA and pinB and which encode a functional protein.

The present invention utilizes nucleic acid molecules encoding puroindoline proteins which hybridize with nucleic acid molecules comprising sequences complimentary to pinA and pinB under conditions of sufficient stringency to produce a clear signal. As used herein, “nucleic acid” is defined as RNA or DNA encoding puroindoline peptides, or are complimentary to nucleic acids encoding such peptides, or hybridize to such nucleic acids and remain stably bound to them under stringent conditions, or encode polypeptides sharing at least 60% sequence identity, or at least 65% sequence identity, or at least 70% sequence identity, or at least 75% sequence identity, or at least 80% sequence identity, or at least 85% sequence identity, preferably at least 90% sequence identity, and more preferably at least 95% sequence identity with the PINA and PINB peptide sequences.

Plants naturally contain wildtype pinA and pinB genes that code for wildtype PINA and PINB, respectively. Wildtype, when referring to nucleic acid sequences or protein sequences, means the genetic constitution of an organism in which a number of mutations (markers) may already exist at the start of a program of mutagenesis before further changes are introduced. Thus, the wildtype PINA and PINB proteins refers to the various forms of the puroindoline proteins found naturally before the introduction of a nucleotide sequence coding for the wildtype pinA and pinB genes.

The present invention further provides fragments of any one of the encoding nucleic acids molecules. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments of the invention encode the lipid binding domains or regions of the puroindolines of the present invention. If the fragment is to be used as a nucleic acid probe or PCR primer, then the fragment length is chosen so as to obtain a relatively small number of false positives during probing and priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al., (1981) J. Am. Chem. Soc. 103, 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

III. Isolation of Other Related Nucleic Acid Molecules

As described herein, the identification and characterization of the nucleic acid molecules encoding a puroindoline or a fragment of a puroindoline allows a skilled artisan to isolate nucleic acid molecules that encode other members of the protein family in addition to the sequences herein described. Further, the presently disclosed nucleic acid molecules allow a skilled artisan to isolate nucleic acid molecules that encode other members of the family of proteins in addition to the puroindolines disclosed herein.

Essentially, a skilled artisan can readily use any one of the amino acid sequences disclosed herein to generate antibody probes to screen expression libraries prepared firm appropriate cells. Typically, polyclonal antiserum from mammals such as rabbits immunized with the purified protein or monoclonal antibodies can be used to probe a cDNA or genomic expression library to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any organism.

Oligomers containing approximately 18-20 nucleotides (encoding about a six to seven amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

IV. Production of Recombinant Proteins Using a rDNA Molecule

The present invention further provides methods for producing a puroindoline protein of the invention using the nucleic acid molecules herein described. In general terms, the production of a recombinant form of a protein typically involves the following steps: First, a nucleic acid molecule is obtained that encodes a puroindoline protein or a fragment of a puroindoline protein. If the encoding sequence is uninterrupted by introns, it is directly suitable for expression in any host. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host-expression system known in the art for use with the nucleic acid molecules of the invention to produce recombinant protein.

V. Puroindoline Proteins

The puroindoline proteins PINA and PINB are wheat endosperm proteins believed to be involved in determining whether wheat grain is soft or hard textured (see, e.g., Rahman et al., 1994, Eur. J. Biochem. 223(3):917-925) (GenBank Accession Numbers S48186, S48187, S48188, CAA56595, CAA56596, CAA56597, CAA56598, AAC60577).

The amino acid sequences for puroindolineA and puroindolineB isolated from wheat (Triticum aestivum) endosperm were determined by Blochet et at. (1993, FEBS 329(3):336-340) (see FIG. 2 of the article) (see also, GenBank Accession Numbers AAB28037 and S36107). The Blochet et al. article and the associated sequences, including FIG. 2 of the article and the GenBank accessions, are specifically incorporated by reference herein in their entirety.

Blochet et al. (1993, supra) speculate on the possible antibacterial and antifungal properties of the puroindolines and the anti-fungal activity of the proteins has been observed in vitro, as noted by Dubreil et al. (Plant Sci., 1998, 138:121-135). However, Gautier et al. (1994, supra) have noted that there is no experimental evidence available to support this assumed function of the puroindolines in seeds. In addition, Tanchak et al. (1998, Plant Sci. 137:173-184) stated the following: Because of their tryptophan-rich domain which shows some similarity to the mammalian peptide, indolicidin, it has been speculated that puroindolines may be membrane-active toxins with antimicrobial activity. In actual fact, the noted similarity between indolicidin and the plant proteins, puroindolines, GSP and oat tryptophanins, is not particularly strong.” (page 182, paragraph bridging columns 1-2) (citations and references omitted).

PinA and pinB are expressed only in wheat endosperm tissue (Gautier et al., 1994). Neither of these proteins is found in leaf tissue of any plants, nor are there apparent homologs of these genes in plant species outside the grass family.

As used herein, a puroindoline protein refers to a protein that has the amino acid sequence encoded by the polynucleotide of PINA and PINB, allelic variants thereof and conservative substitutions thereof that have puroindoline activity. In addition, the polypeptides utilized in the present invention include the proteins encoded by PINA and PINB, as well as polypeptides and fragments, particularly those which have the biological activity of PINA and PINB and also those which have at least 65% sequence identity to the polypeptides encoded by PINA and PINB or the relevant portion, or at least 70% identity, or at least 75% identity, or at least 80% identity, or at least 85% identity to the polypeptides encoded by PINA and PINB or the relevant portion, and more preferably at least 90% similarity to the polypeptides encoded by PINA and PINB or the relevant portion, and still more preferably at least 95% similarity to the polypeptides encoded by PINA and PINB or the relevant portion, and also include portions of such polypeptides. One of skill will recognize whether an amino acid sequence of interest is within a functional domain of a protein, such as the lipid-binding domain of the puroindolines used in the present invention. Thus, it may be possible for a homologous protein to have less than 40% homology over the length of the amino acid sequence but greater than 90% homology in one functional domain.

The puroindoline proteins utilized in the present invention include the specifically identified and characterized variants herein described as well as allelic variants, conservative substitution variants and homologues that can be isolated/generated and characterized without undue experimentation following the methods well known to one skilled in the art.

The term “substantially pure” as used herein refers to puroindoline polypeptides which are substantially free of other proteins, lipids, carbohydrates or other materials with which they are naturally associated. One skilled in the art can purify puroindolines using standard techniques for protein purification.

The invention also utilizes amino acid sequences coding for isolated puroindoline polypeptides. The polypeptides of the invention include those which differ from the exemplified puroindoline proteins as a result of conservative variations. The terms “conservative variation” or “conservative substitution” as used herein denotes the replacement of an amino acid residue by another, biologically similar residue. Conservative variations or substitutions are not likely to change the shape of the polypeptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like. Therefore, all conservative substitutions are included in the invention as long as the puroindoline polypeptides encoded by the nucleotide sequence are functionally unchanged or similar.

As used herein, an isolated puroindoline protein can be a full-length PINA or PINB or any homologue of such proteins, such as puroindoline proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycosylphosphatidyl inositol), wherein modified protein retains the physiological characteristics of natural puroindoline proteins. A homologue of a puroindoline protein is a protein having an amino acid sequence that is sufficiently similar to a natural puroindoline protein amino acid sequence that a nucleic acid sequence encoding the homologue is capable of hybridizing under stringent conditions to (i.e., with) a nucleic acid sequence encoding the natural puroindoline protein amino acid sequence. Appropriate stringency requirements are discussed above.

Puroindoline protein homologues can be the result of allelic variation of a natural gene encoding a puroindoline protein. Natural genes are also referred to as “wildtype genes.” A natural, or wildtype, gene refers to the form of the gene found most often in nature. Puroindoline protein homologues can be produced using techniques known in the art including, but not limited to, direct modifications to a gene encoding a protein using, for example, classic or recombinant DNA techniques to effect random or targeted mutagenesis.

Minor modifications of the PINA and PINB primary amino acid sequence may result in proteins which have substantially equivalent activity as compared to the puroindolines produced by the genes described herein. As used herein, a “functional equivalent” of a puroindoline protein is a protein which possesses a biological activity or immunological characteristic substantially similar to a biological activity or immunological characteristic of non-recombinant, or natural, puroindoline. The term “functional equivalent” is intended to include the fragments, variants, analogues, homologues, or chemical derivatives of a molecule which possess the biological activity of the puroindoline proteins encoded by the genes of the present invention.

The terms “puroindoline proteins”, “PINA proteins” and “PINB proteins” include all naturally occurring allelic variants of these proteins that possess normal puroindoline activity. In general, allelic variants of PINA and PINB proteins will have slightly different amino acid sequence than that specifically encoded by the genes utilized in the present invention but will be able to produce the exemplified phenotypes. Allelic variants, though possessing a slightly different amino acid sequence than those recited above, will posses the ability to produce a phenotype which exhibits the ability to retard, limit or prevent pathogen infection, growth and/or reproduction.

The methods of the present invention can be used by one skilled in the art of plant breeding and plant husbandry to produce crop plants with improved characteristics for pathogen tolerance or resistance.

Applicants further teach methods of recognizing variations in the DNA sequences of pinA and pinB. One method involves the introduction of a nucleic acid molecule (also known as a probe) having a sequence complementary to the puroindoline genes utilized in the invention under sufficient hybridizing conditions, as would be understood by those in the art. Another method of recognizing DNA sequence variation associated with pinA and pinB is direct DNA sequence analysis by multiple methods well known in the art. Another embodiment involves the detection of DNA sequence variation in the puroindolines as represented by different plant genera, species, strains, varieties or cultivars. PinA and pinB can be used as probes to detect the presence of puroindoline genes in other plants. As discussed previously, pinA and pinB sequences have been determined and are readily available to one of ordinary skill in the art. In one embodiment, the sequences will bind specifically to one allele of a puroindoline gene, or a fragment thereof, and in another embodiment will bind to multiple alleles. Such detection methods include the polymerase chain reaction, restriction fragment length polymorphism (RFLP) analysis and single stranded conformational analysis.

Diagnostic probes useful in such assays of the invention include antibodies to PINA and PINB. The antibodies may be either monoclonal or polyclonal, produced using standard techniques well known in the art (See Harlow & Lane's Antibodies: A Laboratory Manual, Cold Spring Harbor Laboratory Press, 1988). They can be used to detect puroindoline proteins by binding to the protein and subsequent detection of the antibody-protein complex by ELISA, Western blot or the like. Antibodies are also produced from peptide sequences of PINA and PINB using standard techniques in the art (See Protocols in Immunology, John Wiley & Sons, 1994). Fragments of the monoclonals or the polyclonal antisera which contain the immunologically significant portion can also be prepared.

Assays to detect or measure puroindoline polypeptides in a biological sample with an antibody probe may be based on any available format. For instance, in immunoassays where puroindoline polypeptides are the analyte, the test sample, typically a biological sample, is incubated with anti-pinA or pinB antibodies under conditions that allow the formation of antigen-antibody complexes. Various formats can be employed, such as “sandwich” assay where antibody bound to a solid support is incubated with the test sample; washed, incubated with a second, labeled antibody to the analyte; and the support is washed again. Analyte is detected by determining if the second antibody is bound to the support. In a competitive format, which can be either heterogeneous or homogeneous, a test sample is usually incubated with an antibody and a labeled competing antigen, either sequentially or simultaneously. These and other formats are well known in the art.

VI. Transformation Methods

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation (see, e.g., U.S. Pat. Nos. 5,405,765, 5,472,869, 5,538,877, 5,538,880, 5,550,318, 5,641,664, and 5,736,369; Watson et al., Recombinant DNA, Scientific American Books (1992); Hinchee et al., Bio/Tech 6:915-922 (1988); McCabe et al., Bio/Tech. 6:923-926 (1988); Toriyama et al., Bio/Tech 6:1072-1074 (1988); Fromm et al., Bio/Tech. 8:833-839 (1990); Mullins et al., Bio/Tech. 8:833-839 (1990); and, Raineri et al., Bio/Tech. 8:33-38 (1990)).

Many of the manipulations being carried out in crop plants are meant for disease and pest resistance, product quality and tolerance to environmental stresses. Dale et al. (1993) reported that there were 395 transgenic plants approved for yield releases in different countries up to 1991. Logemann et al. (1992) have reported the expression of a barley ribosome-inactivating protein which afforded increased protection against R. solani in transgenic tobacco. Song et al. (1995) reported the expression of a receptor kinase-like protein which was encoded by the rice bacterial blight disease resistance gene xa21 in transgenic rice. Lin et al. (1995) have reported the expression of a chitinase gene in transgenic rice plants which showed resistance to the rice sheath blight pathogen, R. solani.

Most approaches have been marginally successful. Clearly, additional transgenic approaches using other antimicrobial genes would be useful.

VII. Transgenes

Genes successfully introduced into plants using recombinant DNA methodologies include, but are not limited to, those coding for the following traits: seed storage proteins, including modified 7S legume seed storage proteins (U.S. Pat. Nos. 5,508,468, 5,559,223 and 5,576,203); herbicide tolerance or resistance (U.S. Pat. Nos. 5,498,544 and 5,554,798; Powell et al., Science 232:738-743 (1986); Kaniewski et al., Bio/Tech. 8:750-754 (1990); Day et al., Proc. Natl. Acad. Sci. USA 88:6721-6725 (1991)); phytase (U.S. Pat. No. 5,593,963); resistance to bacterial, fungal, nematode and insect pests, including resistance to the lepidoptera insects conferred by the Bt gene (U.S. Pat. Nos. 5,597,945 and 5,597,946; Hilder et al., Nature 330:160-163; Johnson et al., Proc. Natl. Acad. Sci. USA, 86:9871-9875 (1989); Perlak et al., Bio/Tech. 8:939-943 (1990)); lectins (U.S. Pat. No. 5,276,269); and flower color (Meyer et al., Nature 330:677-678 (1987); Napoli et al., Plant Cell 2:279-289 (1990); van der Krol et al., Plant Cell 2:291-299 (1990)).

VIII. Expression Units to Express Exogenous DNA in a Plant

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include any plant species.

Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al., (1972) Proc. Natl. Acad. Sci. USA 69, 2110-2114; and Maniatis et al., (1982) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al., (1973) Virology 52, 456-467; and Wigler et al., (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, (1975) J. Mol. Biol. 98, 503-517; or Berent et al., (1985) Biotech. Histochem. 3, 208; or the proteins produced from the cell assayed via an immunological method.

As provided herein elsewhere, several embodiments of the present invention employ expression units (or expression vectors or systems) to express an exogenously supplied nucleic acid sequence, such as the sequence coding for PINA and PINB protein in a plant. Methods for generating expression units/systems/vectors for use in plants are well known in the art and can readily be adapted for use in expressing the puroindoline proteins in a plant cell. A skilled artisan can readily use any appropriate plant/vector/expression system in the present methods following the outline provided herein.

The expression control elements used to regulate the expression of the protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 35S promoter to control gene expression in a plant. Lastly, plant promoters such as prolifera promoter, fruit-specific promoters, Ap3 promoter, heat shock promoters, seed-specific promoters, etc. can also be used. The most preferred promoters will be most active in seedlings.

Either a constitutive promoter (such as the CaMV or Nos promoter), an organ-specific promoter (such as the E8 promoter from tomato) or an inducible promoter is typically ligated to the protein or antisense encoding region using standard techniques known in the art. The expression unit may be further optimized by employing supplemental elements such as transcription terminators and/or enhancer elements.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a preexisting vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J. 3: 835-846 (1984)) or the nopaline synthase signal (Depicker et al., Mol. and Appl. Genet. 1: 561-573 (1982)).

The resulting expression unit is ligated into or otherwise constructed to be included in a vector which is appropriate for higher plant transformation. The vector will also typically contain a selectable marker gene by which transformed plant cells can be identified in culture. Usually, the marker gene will encode antibiotic resistance. These markers include resistance to G418, hygromycin, bleomycin, kanamycin, and gentamicin. After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

The pinA and pinB sequences utilized in the present invention can also be fused to various other nucleic acid molecules such as Expressed Sequence Tags (ESTs), epitopes or fluorescent protein markers.

ESTs are gene fragments, typically 300 to 400 nucleotides in length, sequenced from the 3′ or 5′ end of complementary-DNA (cDNA) clones. Nearly 30,000 Arabidopsis thaliana ESTs have been produced by a French and an American consortium (Delseny et al., FEBS Lett. 405(2):129-132 (1997); Arabidopsis thaliana Database, http://genome.www.staniford.edu/Arabidopsis). For a discussion of the analysis of gene-expression patterns derived from large EST databases, see, e.g., M. R. Fannon, TIBTECH 14:294-298 (1996).

Biologically compatible fluorescent protein probes, particularly the self-assembling green fluorescent protein (GFP) from the jellyfish Aequorea victoria, have revolutionized research in cell, molecular and developmental biology because they allow visualization of biochemical events in living cells (Murphy et al., Curr. Biol. 7(11):870-876 (1997); Grebenok et al., Plant J. 11(3):573-586 (1997); Pang et al., Plant Physiol. 112(3) (1996); Chiu et al., Curr. Biol. 6(3):325-330 (1996); Plautz et al., Gene 173(1):83-87 (1996); Sheen et al., Plant J. 8(5):777-784 (1995)).

Site-directed mutatgenesis has been used to develop a more soluble version of the codon-modified GFP call soluble-modified GFP (smGFP). When introduced into Arabidopsis, greater fluorescence was observed when compared to the codon-modified GFP, implying that smGFP is brighter because more of it is present in a soluble and functional form (Davis et al., Plant Mol. Biol. 36(4):521-528 (1998)). By fusing genes encoding GFP and beta-glucuronidase (GUS), researchers were able to create a set of bifunctional reporter constructs which are optimized for use in transient and stable expression systems in plants, including Arabidopsis (Quaedvlieg et al., Plant Mol. Biol. 37(4):715-727 (1998)).

Berger et al. (Dev. Biol. 194(2):226-234 (1998)) report the isolation of a GFP marker line for Arabidopsis hypocotyl epidermal cells. GFP-fusion proteins have been used to localize and characterize a number of Arabidopsis genes, including geranylgeranyl pyrophosphate (GGPP) (Zhu et al., Plant Mol. Biol. 35(3):331-341 (1997).

IX. Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population which is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection. In mass selection, desirable individual plants are chosen, harvested, and the seed composited without progeny testing to produce the following generation. Since selection is based on the maternal parent only, and their is no control over pollination, mass selection amounts to a form of random mating with selection. As stated above, the purpose of mass selection is to increase the proportion of superior genotypes in the population.

Synthetics, A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous. Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can also be produced in wheat and rice. Hybrids can be formed a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an outbreeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity which results in increased vigor of growth, survival, and fertility of hybrids as compared with the parental lines which were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8: 161-176, In Hybridization of Corp Plants, supra.

X. Materials and Methods

Production of Transgenic Plants

Vectors according to the invention may be used to transform plants as desired, to make plants according to the invention as discussed elsewhere herein.

Rice Transformation. The methods described by Sivamani et al. (1996) has been adopted for transforming rice cultivar ‘M202’ (Johnson et al. 1986). The technique as routinely practiced initially utilizes embryogenic calli cultured from mature seeds.

The Biolistic PDS-1000 He (Bio-Rad laboratories, USA) device was used for transforming the rice tissues via microprojectile bombardment.

For rice calli 1500 psi rupture discs were used. Other procedures such as sterilization of the rupture discs, macrocarriers, stopping screens etc., were strictly in accordance with the manufacturer's manual.

Wheat Transformation. The methods described by Weeks et al. (1993) and Vasil et al. (1993) have been adopted with minor modifications for transforming the wheat cultivar ‘Bobwhite’. The technique as routinely practiced initially utilizes immature embryos isolated from wheat cultivars approximately 7 days post anthesis.

The Biolistic PDS-1000 He (Bio-Rad laboratories, USA) device was used for transforming the wheat tissues via microprojectile bombardment.

For wheat calli 1500 psi rupture discs were used. Other procedures such as sterilization of the rupture discs, macrocarriers, stopping screens etc., were strictly in accordance with the manufacturer's manual.

Tomato Transformation. Dicotyledonous plants, such as the tomato, may also transformed to produce plants transgenic for one or more of the puroindoline genes.

For example, tomato plants may be transformed using Ti plasmid technology, for example as described by Bevan (1984) Nucleic Acids Research 12:8711-721 and U.S. Pat. No. 5,141,870. For additional information on the transformation of tomato plants, see, for example, McGurl et al., 1994, Proc. Natl. Acad. Sci. USA 91(21):9799-9802; Tieman et al., 1992, Plant Cell 4:667-679; and McGarvey et al., 1995, Biotechnology 13(13):1484-1487.

As a specific example, transgenic tomato plants can be produced using the binary vector pAGS152 in A. tumefaciens strain LBA4404, wherein the vector contains one or more of the puroindoline genes (U.S. Pat. No. 5,141,870, hereby incorporate by reference in its entirety).

Plasmids

Plasmid construction followed the procedures provided in Hogg et al. (2004), which is incorporated herein in its entirety. Basically, immature embryos from the cultivar ‘Hi-Line’ were transformed and regenerated as described by Beecher et al. (2002).

Callus tissue was bombarded with pGA1.8, pGB4.20, and pRQ101A construct DNA in a 2.5:2.5:1 molar ratio respectively to obtain HGAB lines with added pGA1.8 and pGB4.20 and with a 5:1 molar ratio of pGA1.8:pRQ101A to obtain HGA lines with added pGA1.8. The HGB lines with added pGB4.20 have been described previously (Beecher et al. 2002; see, FIG. 1).

The pina expression vector, pGA 1.8. was made by replacing the glutenin coding region of the pGlu10(5) construct (Blechl and Anderson 1996) with the ‘soft-type’ pina coding sequence amplified from ‘Chinese Spring’ genomic DNA. The ‘soft type’ pina sequence is under the control of the glutenin regulatory elements Dy10 (5′) and Dx5 (3′). The pinb expression vector pGB4.20 (Beecher et al. 2002) was made in a the same way except that the ‘soft type’ pinb coding sequence from ‘Chinese Spring’ was used.

These plasmids were constructed using the intact pinA (GenBank Accession No. X69913 or pinB (GenBank Accession No. X69912; SEQ ID NO: 3) coding sequences. Constructs include transit peptides and consensus start site.

Primers used for amplifying pinA coding sequences using the RT-PCR technique were PA5BH (5′ CGGGATCCAACAATGAAGGCCCTCTTCCTCATAGG 3′) (SEQ ID NO. 7) and PA3BH (5′ GGATCCCGCCAGTAATAGCCAATAGTGCCGGGGAT 3′) (SEQ ID NO. 8).

Primers used for amplifying pinB coding sequences using the RT-PCR technique were PB5BH (5′ CGGGATCCAACAATGAAGACCTTATTCCTCCTAGC 3′) (SEQ ID NO. 9) and PA3BH (5′ GGATCCCGCCAGTAATAGCCACTAGGGAACTT 3′) (SEQ ID NO. 10).

A sample of RNA prepared from the variety Chinese Spring was used as source material for the RT-PCR reaction as per standard techniques. The amplified genes were digested by BamHI and ligated into a vector downstream of the ubiquitin promoter. The ubiquitin promoter construct had been previously digested with BamHI and phosphatase treated to prevent self ligation. pPuroA and pPuroB plasmids used in transformation experiments were verified by standard sequencing techniques to assure no errors had occurred in the PCR amplification.

Selection and Regeneration of Transgenic Plants

Rice and Wheat. Transgenic rice plants were obtained from the bombarded embryogenic calli of rice by the technique of Sivamani et al. (1996) using hygromycin selection.

Transgenic wheat plants were obtained from bombarded immature embryos by the methods described by Weeks et al. (1993) and Vasil et al. (1993) using bialaphos (Meiji Seika Kaisha Ltd, Japan) selection.

The resistant calli of rice and wheat were transferred to medium to induce production of both shoots and roots. Putative transgenic plantlets were transferred to the greenhouse and allowed to self-fertilize. For wheat, typically more than 75% of these plantlets are escapes and true transgenic plants were selected by spraying the plants with 0.1% glufosinate (Liberty®, Agrevo Inc.).

Tomato. Transgenic tomato plants can be obtained by agroinoculation by the technique of Day et al. (1991, Proc. Natl. Acad. Sci. USA 88(15):6721-6725) using hygromycin selection. Alternative methods of selecting the transformed tomato plants include kanamycin resistance selection and xylose isomerase selection (Haldrup et al., 1998, Plant Mol. Biol. 37(2):287-296).

### EXAMPLES

### Example 1

### Dry and Wet Milling

Hard/Soft NILs. The first group of isolines consisted of two sets of hard/soft NILs chosen from those summarized by Morris and Allen (2001) and Morris et al (2001a). The first set of NILs were the Australian white spring cultivar ‘Falcon’ derived NILs that carried either the Pina-D1a soft type allele derived from ‘Heron’ or the ‘Falcon’ derived Pina-D1b hard type Pina null allele. Both ‘Falcon’ and ‘Heron’ contain the Pinb-D1a soft type Pinb allele (Giroux and Morris, 1998) (Table I). Two accessions of hard type ‘Falcon’ (PI 612556 and PI 612554) and two accessions of soft type ‘Falcon’ (PI 612555 and PI 612553) formed the first set of NILs. The second set of NILs consisted of sibling accessions of ‘Gamenya’, another Australian cultivar classified as hard ‘Gamenya’ (accessions PI 612548 and PI 612552) carrying the Pina null mutation (Pina-D1b) and soft ‘Gamenya’ (accessions PI 612549 and PI 612551) carrying the functional Pina-D1a allele (Table I). All ‘Gamenya’ lines contain soft type Pinb (Pinb-D1a). Seeds from this group of genetic material were obtained from single row plots grown at the Arthur H. Post Field Research farm near Bozeman, Mont. under irrigated condition. Each plot was a 3-m row seeded with 4 g with row spacing of 30 cm. Plots received 7.6 cm of water 1 wk before and 1 wk after anthesis. At maturity, plots were cut with a binder (Mitsubishi Agricultural Machinery Co; Ltd, Tokyo, Japan), threshed with a Vogel bundle thrasher (Bill's Welding, Pullman, Wash.), cleaned, and weighed.

Puroindoline Overexpressing Transgenic Isolines. The second group of genetic material included ‘Hi-line’ (Lanning et al 1992), a hard red spring wheat cultivar carrying a glycine-to-serine change in the tryptophan-rich domain of PINB (Pinb-D1b) (Giroux and Morris 1997) and a soft type Pina (Pina-D1a), and a selected subset of transgenic lines created in the ‘Hi-Line’ background by adding wild type Pina, Pinb or both under the control of glutenin regulatory elements Dy10 (5′) and Dx5 (3′) (Hogg et al 2004). In this subset the transgenic line with added Pina (HGA3), Pinb (HGB12), and both Pina and Pinb (HGAB18) had intermediate, soft and very soft grain texture, respectively (Table I). These four genotypes were grown in 2004 in two replications of a randomized block design under both rainfed and irrigated conditions in 12 row plots 25.6 m long for the rainfed trial and 15.2 m long for the irrigated trial, with rows 30 cm apart at the Arthur H. Post Field Research farm near Bozeman, Mont. (Martin et al 2007). Each plot was harvested with a plot combine, cleaned, and weighed.

Grain Characterization and Dry Milling. Kernel hardness and seed weight were determined using the Single Kernel Characterization System (SKCS) 4100 (Perten Instruments, Springfield, Ill.) (Table I). One hundred-fifty g of ‘Falcon’ NILs and ‘Gamenya’ isogenic siblings were milled on a Brabender Quadrumat Jr. flour mill (Brabender GmbH, Duisburg, Germany) as described by Campbell et al (2007). The seeds were tempered to 14% moisture content for the soft NILs and to 15.5% for the hard NILs, conditioned for 24 hr as per Approved Method 26-50 (AACC, 2003). Flour and bran weights were measured and total flour yield was calculated as (grams of flour)/(grams of flour and bran).

Dry milling of ‘Hi-Line’ and the transgenic isolines was done as previously described (Martin et al 2007) using a Miag Multomat pilot scale mill. The mill produced 10 flour streams and four feed streams form three break and five reduction rolls. Straight grade flour was used in our experiment.

Wet Milling. The wet milling determinations were completed on two independent extractions for ‘Falcon’ and ‘Gamenya’ derived NILs and on three independent extractions for ‘Hi-Line’ and soft transgenic isolines. Wet milling of flour was done by a dough-dispersion and centrifugation method (Sayaslan 2006), an adapted method from Czuchajowska and Pomeranz (1993), with some minor modifications. Flour (50-75 g, 14% mb) was mixed in a ML-33777 N50 Hobart mixer (Troy Ohio) with gradual addition of water (45-50 ml, 25° C.) until the mixture became a cohesive, stiff dough, and cleaned itself from the mixing bowl (3-4 min). The developed stiff dough was covered with 150 ml water at 25° C. and was rested at room temperature for 30 min. The dough and liquid was transferred to a blender (TSK-9368AP, China) and dispersed at high speed for 1 min. The slurry was transferred to 250 ml Sorvall centrifuge bottles and centrifuged at 2500×g at 25° C. for 15 min using a Sorvall super T21 centrifuge (Kendro Laboratory Products, Newtown, Conn.), then the supernatant was weighed and discarded. The top layer which consisted mainly of gluten, insoluble pentosanis, damaged starch, and small granular starch, was carefully removed from the bottom layer which consisted of primary prime starch. The primary prime starch was weighed and dried for 2 days at 37° C. using a forced air incubator. The gluten was hand manipulated in a beaker under three consecutive 150 ml water washes to obtain the cohesive wet gluten and starch milk. The wet gluten was partly frozen (˜30 min at −20° C.), cut into ˜2 cm³ pieces and incubator dried for 3 days at 37° C. The starch milk obtained from gluten washing was collected and centrifuged at 2500×g at 25° C. for 15 min. The supernatant from the second centrifugation was discarded and the top partly pigmented layer (tailing) was separated from the secondary prime starch. Both fractions were incubator dried for 2 days at 37° C. The dried fractions obtained from each independent extraction were coarsely ground using a mortal and pestle and then ground using a Perten Laboratory Mill 3303 (Perten Instruments, Springfield, Ill.).

Moisture, starch and protein contents of the flours and the dried wet milling products were measured after recording dry weights of each fraction. Moisture was determined by AACC Method 44-15A (AACC, 2003). Starch content was measured by a total starch assay (AACC Method 76-13, 2003) using a Megazyme kit (Megazyme International. Bray, Co Wicklow, Ireland), Protein content (N×5.7) was measured using a Leco FP-2000 (Leco Corp; St. Joseph, Mich.). Both starch and protein percentages were converted to a dry weight basis after determining as-is moisture content.

Data Analyses. All variables from wet milling were analyzed via analysis of variance using PROC GLM in SAS ((SAS Institute, Inc; Cary, N.C.). For the ‘Hi-Line’ and transgenic isolines the model was analogous to a randomized block split plot combined over environments where genotypes were main plots and independent extractions were subplots. Comparisons between genotypes were made using LSD. The hard/soft near isogenic pairs were analyzed using a model that accounted for independent extractions, genotypes (cultivars×hard vs. soft combination), and sibs within each genotype. Comparisons were made between hard and soft for each cultivar and averaged over cultivars.

Results. Two sets of soft/hard NILs differing in Pina function and grain hardness were used for dry and wet milling. The first set consisted of ‘Falcon’ derived soft/hard NILs with two soft accessions carrying Pina-D1a and two hard accessions carrying the Pina-D1b allele; the second set consisted of soft/hard ‘Gamenya’ NILs consisting of two soft Pina-D1a and two hard Pina-Dub accessions (Morris and Allen 2001; Morris et al 2001a) (Table I). All ‘Falcon’ and ‘Gamenya’ NILs have the soft-type Pinb allele (Pinb-D1a). Single kernel characterization system (SKCS) grain hardness was 82 in hard ‘Falcon’ and ‘Gamenya’ NILs and 29 and 37.5 for soft ‘Falcon’ and soft ‘Gamenya’ NILs, respectively. The ‘Falcon’ NILs had higher kernel weight than the ‘Gamenya’ NILs (Table I).

The second group of the genetic material used for starch extractability assay was the transgenic isolines used by Martin et al (2007) in a study assessing the effect of grain hardness on pilot scale milling quality. This subset included ‘Hi-Line’ hard red spring wheat, ‘Hi-Line’ transgenic isolines overexpressing PINA (HGA3), PINB (HGB12) or both PINA and PINB (HGAB18) (Table I). Grain texture ranged from very soft (HGAB18) to hard (‘Hi-Line’) (Table I). Kernel weight was higher in HGB12 and ‘Hi-Line’ than HGAB18 and HGA3 respectively (Table I).

TABLE I
Kernel characteristics and puroindoline genotypes of soft/hard ‘Falcon’ and
‘Gamenya’ near isogenic lines and ‘Hi-Line’ transgenic isolines.

| | | | | Kernel |
|---|---|---|---|---|
| | | Added Pin coding | SKCS grain | weight ^{c} |
| Genotype | Native pin ^{a} | sequence ^{b} | hardness ^{c} | (mg) |
| Hard Falcon ^{d} | Pina-D1b/Pinb-D1a | | 82 | 34.6 |
| Soft Falcon ^{d} | Pina-D1b/Pinb-D1a | | 29 | 33.5 |
| Hard Gamenya ^{d} | Pina-D1b/Pinb-D1a | | 82 | 29.5 |
| Soft Gamenya ^{d} | Pina-D1b/Pinb-D1a | | 38 | 26.6 |
| P value ^{e} | | | <.0001 | 0.0779 |
| P value ^{f} | | | <.0001 | 0.007 |
| CV % | | | 4.3 | 3.8 |
| LSD(0.05) | | | 6.9 | 3.3 |
| Hi-Line | Pina-D1a/Pinb-D1b | | 73.7 | 35.0 |
| HGA3 | Pina-D1a/Pinb-D1b | Pina-D1a | 42.0 | 33.6 |
| HGB12 | Pina-D1a/Pinb-D1b | Pinb-D1a | 9.7 | 35.4 |
| HGAB18 | Pina-D1a/Pinb-D1b | Pina-D1a/Pinb-D1a | 6.4 | 34.3 |
| P value ^{f} | | | 0 | 0.007 |
| CV % | | | 5.8 | 1.4 |
| LSD(0.05) | | | 3.3 | 0.8 |

^{a} Native pin refers to the wild-type Pin allele residing at the Ha locus. Pina-D1b and Pinb-D1b contain a null and glycine-serine mutations, respectively while Pina-D1a and Pinb-D1a are the soft type functional alleles.
^{b} Added pin is the coding sequence from the allele or alleles used to transform Hi-line hard red spring wheat (Hogg et al 2005).
^{c} Kernel characteristics for Falcon and Gamenya NILs were determined using the Single Kernel Characterization System. Data for Hi-Line and transgenic isolines were taken from Martin et al (2007).
^{d} The values for these genotypes were averaged over two lab replications and two accessions for each genotype planted in irrigated environment.
^{e} P value for the average of soft vs. hard NILs.
^{f} Genotype main effect P value.

The flour yield from the hard ‘Falcon’ and ‘Gamenya’ NILs were higher than their soft isolines, and that relationship was also seen in the transgenic isolines in which hardness was positively correlated with flour yield (Table II). Flour protein was higher in hard than soft ‘Gamenya’ NILs. This trend was opposite for ‘Falcon’ NILs. For the ‘Hi-Line’ isolines, flour protein was highest for the untransformed control variety ‘Hi-Line’ and lowest in HGB12. Flour starch was not significantly related to hardness for any of the comparison groups (Table II).

TABLE II
Flour yield and flour protein and starch content for
soft/hard ‘Falcon’ and ‘Gamenya’ near isogenic
lines and ‘Hi-Line’ transgenic isolines.

| | Flour | Flour | Flour |
|---|---|---|---|
| | yield ^{a} | protein ^{b d} | starch ^{c d} |
| Genotype | g kg⁻¹ | | |
| Hard Falcon ^{e} | 741 | 141 | 747 |
| Soft Falcon ^{e} | 706 | 143 | 755 |
| Hard Gamenya ^{e} | 724 | 158 | 762 |
| Soft Gamenya ^{e} | 710 | 151 | 798 |
| P value ^{g} | 0.051 | 0.480 | 0.233 |
| P value ^{h} | 0.149 | 0.047 | 0.258 |
| CV % | 1.8 | 2.7 | 2.9 |
| LSD(0.05) | 37 | 11 | 63 |
| Hi-Line ^{f} | 744 | 155 | 734 |
| HGA3 ^{f} | 732 | 153 | 735 |
| HGB12 ^{f} | 714 | 149 | 738 |
| HGAB18 ^{f} | 711 | 152 | 768 |
| P value ^{h} | 0 | <.0001 | 0.322 |
| CV % | 1 | 0.5 | 3.7 |
| LSD(0.05) | 11 | 1.3 | 48 |

^{a} Flour yield for Falcon and Gamenya isogenic lines obtained by a Brabender Quadrumat Jr. flour mill and straight grade flour yield for Hi-Line and transgenic isolines obtained by Martin et al 2007 using a Miag Multomat pilot scale mill.
^{b} Flour protein was obtained using a Leco FP-2000 Nitrogen Determinator.
^{c} Flour starch was obtained by Megazyme Amyloglucosidase/α-Amylase method using Megazyme kit.
^{d} These means are on a dry basis.
^{e} The values for these genotypes were averaged over two accessions for each genotype planted in irrigated environment.
^{f} The values for these genotypes were averaged over two field replications for rainfed and irrigated environments at Bozeman, MT.
^{g} P value for the average of soft vs. hard NILs.
^{h} Genotype main effect P value.

All flours were then fractionated via a wet milling dough-dispersion and centrifugation procedure (Czuchajowska and Pomeranz 1993; Sayaslan 2006). The wet milling procedure resulted in the recovery of the tour main fractions primary prime starch, secondary prime starch, gluten, and tailings. Primary prime starch resulted from the first centrifugation of the slurry. Separation of the phases after centrifuge gave supernatant, unwashed gluten (gluten with adhering starch, the tailings (mainly cell walls and pentosans), and primary prime starch fractions. The mean yield (dry weight) of all combined fractions separated from the four NILs is given in Table III. For the ‘Falcon’ and ‘Gamenya’ NILs, the soft NILs had higher total prime starch yields in both genetic backgrounds than their hard isolines, however the difference in total prime starch yield was not significant for ‘Falcon’, but approached significance for ‘Gamenya’ (P=0.06) and when averaged over both genetic backgrounds (P=0.055). Gluten yield was not different between soft and hard NILs in either the ‘Falcon’ or ‘Gamenya’ background however in both backgrounds, the trend toward soft NILs having increased gluten content was evident (Table III). Tailing fraction yield was significantly higher in hard than soft NILs. Total product recovery percentage difference was non-significant between soft and hard ‘Falcon’ NILs but was significant in the ‘Gamenya’ background in which the soft NILs had higher total product recovery relative to the hard NILs (77 vs 72%). Given the trend toward increased starch yield in soft NILs in both the ‘Falcon’ and ‘Gamenya’ backgrounds, we explored whether higher transgenically conditioned PIN levels would improve starch extractability relative to a hard control.

Wet milling yield of ‘Hi-Line’ and three transgenic isolines is given in Table III. Genotypes differed for yield of primary prime starch, total prime starch and gluten. Yields of total prime starch were higher in all three transgenic isolines overexpressing one or both PINs relative to ‘Hi-Line’ (P<0.0001) with the highest yield in the intermediate hardness line HGA3. The soft and supersoft textured lines, HGB12 and HGAB18, were not significantly different from each other in starch yield. Interactions between genotype and environment were not detected for total prime starch yield. Although lab replications showed significant differences in total prime starch yields (P<0.0001), genotype interaction with lab replications was not detected (P=0.243). The gluten yield was significantly reduced in HGA3 relative to untransformed ‘Hi-Line’, HGB12, and HGAB18. Environment effect was highly significant for dry gluten yield (P=0.0066) with flour milled from seed grown under rainfed conditions having more gluten yield than flour milled from seed grown under the irrigated environment (218.7 vs. 194.1 g kg-b), but no interaction effect was detected between genotype and environment (P=0.824). Lab replications did not show any significant effect on gluten extractability (P=0.149). Neither genotype (Table III) nor environment had a significant effect on tailing yield. The weight of this fraction showed differences among lab replications (P<0.0001), but there was no interaction effect between genotype and lab replication for tailing yield (P=0.666). The recovery percentage of total extracted fractions showed significant difference among genotypes with ‘Hi-Line’ having the lowest recovery percentage. The recovery percentage didn't follow the same trend that was observed for total prime starch. The softest genotypes (HGAB18 and HGB12) showed higher recovery compared to HGA3, due to their higher yield of gluten. Genotype by environment effect was not detected for total recovery percentage, but rainfed samples had higher recovery percentage than irrigated samples (0.752 vs. 0.735% with a P value of 0.0034). In fact the higher total recovery percentage for rainfed versus irrigated environment is a consequence of environmental effect on gluten yield extraction. The higher gluten extractability from rainfed versus irrigated samples is directly related to the environment effect on the flour protein (145 and 152 g kg⁻¹ protein content obtained from irrigated and rainfed respectively with a P value of <0.0001).

TABLE III
Yield data (dry basis) for wet milling fractions from flour of soft/hard ‘Falcon’ and
‘Gamenya’ near isogenic lines and ‘Hi-Line’ transgenic isolines.

| | Primary | Secondary | Total | | | | |
|---|---|---|---|---|---|---|---|
| | prime | prime | prime | | | Total | Starch |
| | starch | starch | starch | Gluten | Tailing | Recovery | Recovery |
| Genotype | g kg⁻¹ | | | | | % | % |
| Hard Falcon ^{a} | 291.5 | 129 | 420.5 | 174.5 | 109 | 0.70 | 56.3 |
| Soft Falcon ^{a} | 340 | 103 | 443 | 195 | 82.5 | 0.72 | 58.7 |
| Hard Gamenya ^{a} | 259 | 118 | 377 | 224.5 | 123.5 | 0.72 | 49.5 |
| Soft Gamenya ^{a} | 315 | 111 | 426 | 238.5 | 103 | 0.77 | 53.4 |
| P value ^{c} | 0.011 | 0.086 | 0.045 | 0.121 | 0.002 | 0.117 | 0.045 |
| P value ^{d} | 0.036 | 0.007 | 0.070 | 0.009 | 0.005 | 0.127 | 0.070 |
| CV % | 10 | 14 | 7 | 9 | 9 | 4 | 7 |
| LSD(0.05) | 51.3 | 27.6 | 48.9 | 32.7 | 17 | 0.05 | 2.35 |
| Hi-Line ^{b} | 304.3 | 130.2 | 434.5 | 203.8 | 83.5 | 0.72 | 59.2 |
| HGA3 ^{b} | 367.1 | 117.4 | 484.5 | 186.3 | 73.4 | 0.74 | 65.92 |
| HGB12 ^{b} | 365.6 | 105 | 471.6 | 210.5 | 72.3 | 0.75 | 63.90 |
| HGAB18 ^{b} | 336.3 | 128.3 | 464.7 | 218.2 | 71 | 0.75 | 60.5 |
| P value ^{d} | 0.026 | 0.407 | <.0001 | 0.025 | 0.263 | 0.017 | 0.017 |
| CV % | 15 | 33 | 3.9 | 11 | 22 | 3.4 | 3.4 |
| LSD(0.05) | 41.9 | 38.3 | 17 | 18.6 | 9.51 | 0.01 | 0.85 |

^{a} The values for these genotypes were averaged over two lab replications and two accessions for each genotype planted in irrigated environment.
^{b} The values for these genotypes were averaged over 3 lab replications and two field replications for rainfed and irrigated environments at Bozeman, MT.
^{c} P value for the average of soft vs. hard NILs.
^{d} Genotype main effect P value.

The starch content (dry basis) of fractions separated from the ‘Falcon’ and ‘Gamenya’ NILs are given in Table IV. Starch content of primary and secondary prime starch was not different between hard and soft NILs within and between backgrounds. However, the soft ‘Falcon’ NILs gluten was higher in starch content than gluten extracted from the hard ‘Falcon’ NILs but the hard and soft ‘Gamenya’ NILs did not differ for this trait. The starch content in tailing fraction obtained from hard ‘Falcon’ NILs was higher than in the their counterparts. The mean for starch content of primary and secondary prime starch separated from ‘Hi-Line’ and its transgenic isolines was not different among genotypes, although genotype was a significant effect on starch content of gluten and tailing fractions for this group. Gluten and tailing fraction starch content was lowest in HGA3. The tailing fraction obtained from ‘Hi-Line’ had higher amount of starch than all three transgenic isolines (Table IV).

TABLE IV
Starch content (dry basis) of fractions obtained from wet milling
of flours from soft/hard ‘Falcon’ and ‘Gamenya’ near
isogenic lines and ‘Hi-Line’ transgenic isolines.

| | Primary | Secondary | | |
|---|---|---|---|---|
| | prime | prime | | |
| | starch ^{a} | starch ^{a} | Gluten ^{a} | Tailing ^{a} |
| Genotype | g kg⁻¹ | | | |
| Hard Falcon ^{b} | 993.2 | 985.6 | 383.4 | 814.2 |
| Soft Falcon ^{b} | 964.5 | 980.3 | 476.1 | 763.2 |
| Hard Gamenya ^{b} | 1000.0 | 988.2 | 449.8 | 865.8 |
| Soft Gamenya ^{b} | 999.6 | 974.7 | 470.5 | 865.4 |
| P value ^{d} | 0.347 | 0.437 | 0.019 | 0.110 |
| P value ^{e} | 0.447 | 0.845 | 0.032 | 0.003 |
| CV % | 4 | 2.3 | 8.7 | 3.5 |
| LSD(0.05) | 64.4 | 37.6 | 62.8 | 46.6 |
| Hi-Line ^{c} | 957.3 | 871.3 | 385.2 | 774.7 |
| HGA3 ^{c} | 993.7 | 765.1 | 331.2 | 708 |
| HGB12 ^{c} | 978.0 | 853.7 | 408.9 | 728.7 |
| HGAB18 ^{c} | 980 | 852 | 429 | 719.2 |
| P value ^{e} | 0.291 | 0.208 | 0.0003 | 0.0087 |
| CV % | 4.6 | 15 | 11 | 5.9 |
| LSD(0.05) | 89.9 | 211.5 | 99 | 58.7 |

^{a} Determined by Megazyme Amyloglucosidase/α-Amylase method using Megazyme kit.
^{b} The values for these genotypes were averaged over two lab replications and two accessions for each genotype planted in irrigated environment.
^{c} The values for these genotypes were averaged over 3 lab replications and two field replications for rainfed and irrigated environments at Bozeman, MT.
^{d} P value for the average of soft vs. hard NILs.
^{e} Genotype main effect P value.

The mean protein content (dry basis) of fractions separated from the ‘Falcon’ and ‘Gamenya’ NILs is given in Table V. Although the differences in protein content of primary and secondary prime starch were not significant, primary prime starch fraction obtained from soft NILs had higher protein content than hard NILs. In addition, the soft version of ‘Falcon’ had lower protein content gluten than the hard counterpart, and the ‘Gamenya’ NILs showed the same trend. The amount of protein present in tailing fractions differed significantly in both ‘Falcon’ and ‘Gamenya’ backgrounds in that in each case soft NILs had significantly more protein present in the tailing fraction. Protein content of primary prime starch separated from ‘Hi-Line’ and ‘Hi-Line’ transgenic isolines was not significantly different among genotypes even though all transgenics trended higher in protein content (Table V). Similarly, protein content of secondary prime starch and tailing fractions from the three transgenics were substantially higher than ‘Hi-Line’.

TABLE V
Protein content (dry basis) of fractions obtained from wet milling
of flours for soft/hard ‘Falcon’ and ‘Gamenya’ near
isogenic lines and ‘Hi-Line’ transgenic isolines.

| | Primary | Secondary | | |
|---|---|---|---|---|
| | prime | prime | | |
| | starch ^{a} | starch ^{a} | Gluten ^{a} | Tailing ^{a} |
| Genotype | g kg⁻¹ | | | |
| Hard Falcon ^{b} | 8.1 | 5.1 | 513.8 | 47.4 |
| Soft Falcon ^{b} | 8.6 | 7.9 | 446.5 | 88.4 |
| Hard Gamenya ^{b} | 5.5 | 4.6 | 473 | 32.2 |
| Soft Gamenya ^{b} | 6.6 | 6.1 | 458.9 | 39 |
| P value ^{d} | 0.2304 | 0.1166 | 0.0661 | 0.0081 |
| P value ^{e} | 0.02 | 0.304 | 0.15 | 0.002 |
| CV % | 16 | 41 | 8.1 | 26 |
| LSD(0.05) | 1.89 | 4.01 | 62.4 | 22.3 |
| Hi-Line ^{c} | 6.7 | 3.9 | 535 | 23.9 |
| HGA3 ^{c} | 7.6 | 8.1 | 579.9 | 59.8 |
| HGB12 ^{c} | 7 | 7 | 502 | 58.4 |
| HGAB18 ^{c} | 8 | 6.1 | 489.8 | 52.3 |
| P value ^{e} | 0.229 | 0.0044 | 0.0007 | 0.0011 |
| CV % | 22 | 39 | 8.5 | 40 |
| LSD(0.05) | 2.02 | 3.91 | 69.2 | 23.5 |

^{a} Determined by Leco FP-2000 Nitrogen Determinator.
^{b} The values for these genotypes were averaged over two lab replications and two accessions for each genotype planted in irrigated environment.
^{c} The values for these genotypes were averaged over 3 lab replications and two field replications for rainfed and irrigated environments at Bozeman, MT.
^{d} P value for the average of soft vs. hard NILs.
^{e} Genotype main effect P value.

Czuchajowska and Pomeranz (1993) extracted starch from a set of randomly selected soft and hard wheat varieties. They found the yield of prime starch was higher from the low protein soft wheat flours than from the high-protein hard wheat flours. In order to test whether this difference in starch extractability is linked to the Ha locus on the short arm of chromosome 5D and to Pin expression, we used two sets of soft/hard NILs differing in Pina function and grain hardness and transgenic Pin isolines varying in grain hardness and Pina and/or Pinb expression level. The soft/hard NILs were created in either the ‘Falcon’ or ‘Gamenya’ varieties and in both cases we used two hard type Pina null lines (Pina-D1b) and two soft type Pina functional allele (Pina-D1a) lines (Morris and Allen 2001; Morris et al 2001a) (Table I). The Ha locus did affect total prime starch with the soft having more than the hard counterparts. The Ha locus had no effect on starch or protein content of primary or secondary prime starch fractions in either genetic background (Tables IV and V respectively). This fact along with the lack of any significant differences in the starch content of flour between NILs in either genetic background (Table II) implies that the Ha locus directly impacts starch extractability in these NILs since starch recovery is increased while tailings are decreased. Since tailing starch content is not different between hard and soft NILs (Table IV) (P=0.11) the differences in starch extractability is not because of the starch loss via tailing fractions in hard lines. In order to determine the effect of added PIN and grain softness upon starch yield, we selected the transgenic isolines used by Martin et al (2007) which vary markedly in Pin expression level and grain hardness. All three transgenics had increased recovery of starch with the highest yield seen in the intermediate textured line HGA3 relative to ‘Hi-Line’. Significantly, starch content of recovered prime starch fractions was not decreased in the transgenics and gluten starch content was significantly lower in HGA3 relative to ‘Hi-Line’. The increased starch extractability seen in HGA3 versus ‘Hi-Line’ appears to result from greater separation of starch and gluten in that starch content of gluten and tailings are reduced (Table IV) while protein content of both gluten and tailings are increased in HGA3 relative to ‘Hi-Line’ (Table V).

Wheat grain hardness is most likely determined by the degree of adhesion between starch granules and protein matrix. Friabilin (PINA and PINB) may decrease grain hardness via reduction of the close adherence between starch granule and gluten (Anjum and Walker 1991). Greater separation of starch and gluten in softer textured wheats is likely achieved via puroindolines coating of starch granules and thus preventing tight adhesion between starch granules and the surrounding protein matrix (Swan et al 2006). Starch loss through supernatant may be an explanation for less starch extractability from ‘Hi-Line’ than softer genotypes. Higher starch damage in hard wheat than soft wheat due to milling has been reported (Symes 1965; Letang et al 2001; Van Der Borght et al 2005). The proportion of damaged starch for ‘Hi-Line’ and the three transgenic isolines used in this study was measured in Martin et al (2007). The hard textured ‘Hi-Line’ had the most starch damage followed by the intermediate-textured HGA3, and soft-textured HGB12 and HGAB18. Damaged starch granules have been mentioned as part of squeegee starch that reduce prime starch yield in wet milling (Van Der Borght et al 2005). Moreover damaged starch fraction increases both water absorption (up to three times) (Van Der Borght et al 2005; Tester et al 2006) and endogenous B-amylase hydrolysis, which generates maltose which is useful in the baking industry (Tester et al 2006). Unlike native starch granules, which are semicrystalline, insoluble and as a consequence inaccessible to hydrolysis, damaged starch fragments are amorphous, soluble and readily hydrolysed (Tester et al 2006). The increased water-solubility and susceptibility to hydrolysis due to the level of starch damage may explain the higher rate of starch loss through supernatant in wet milling process of hard wheat versus softer genotypes.

Significant differences among genotypes for tailing yield was not detected (Table III), but tailing fraction obtained from ‘Hi-Line’ showed higher starch content and lower protein content than transgenic isolines (Table IV and V respectively). The presence of starch in tailing can be a result of the admixture of secondary prime starch with tailing pentosans during last phase separation. Small starch granules are admixed with pentosans in tailing, whereas large starch granules make the pure starch fractions (Czuchajowska and Pomeranze 1993). The higher rate of starch loss through admixing with tailing fractions in ‘Hi-Line’ hard wheat may be considered as an explanation for differences in starch extractability between ‘Hi-Line’ and softer transgenic isolines. There are some evidences that weaken the role of tailing in starch loss. First, secondary prime starch is a small portion of total starch. Second, there were significant differences in primary prime starch yield between ‘Hi-Line’ and softer textured isolines. And third, the secondary prime starch obtained from genotypes did not show significant differences in starch content, the expected result if there were significant differences in the admixture between secondary prime starch and tailing due to the phase separation procedure.

As demonstrated above, grain hardness is a significant factor in determining the suitability of wheat for wet milling processes. This effect was observed in hard and soft NILs that differed in the state of their Ha locus, and also in transgenic isolines having increased dosage of Pina and/or Pinb. The overexpression of functional Pins leads to an increase in starch extractability and increased total recovery with highest yields seen in the transgenic line having intermediate grain texture.

### Example II

### Puroindoline Co-localize to the Starch Granule Surface and Increase Starch Polar Lipid Content

Genetic Materials. The genotypes used in this study are a subset of the recombinant lines described by Wanjugi et al. (2007a) and were developed by crossing one transgenic isoline overexpressing PINA (HGA3) or PINB (HGB12) created in the variety ‘HiLine’ (“HL”) (Lanning et al., 1992) and described by Hogg et al. (2004) to either a PINB null, ‘Canadian Red’ (“CR”); hard white spring (Clark, 1926) or a PINA null, ‘McNeal’ (“McN”); hard red spring (Lanning et al., 1995) variety. The transgenic events were selected from the events described by Hogg et al. (2004, 2005) as having good plant vigor and relatively unaltered plant yield, seed size, and seed protein content. ‘Canadian Red’, also referred to as “CR” herein, has the soft type Pina-D1a and a mutant Pinb-D1e allele (Morris et al., 2001). Pinb-D1e contains a point mutation (TGG-TGA) leading to a change in residue Trp-39 to a stop codon. ‘McNeal’, also referred to as “McN” herein, possesses the soft type Pinb-D1a and a mutant Pina allele (Pina-D1b) which is an apparent deletion of the Pina coding sequence (Giroux and Morris, 1998). ‘Hi-Line’, also referred to as “HL” herein, has the soft type Pina-D1a and the mutant Pinb-D1b allele which contains a single point mutation in Pinb resulting in a glycine to serine substitution at the 46^{th} residue of PINB (Giroux et al., 2000). The ‘Hi-Line’ derived transgenic lines (HGA3, HGB12) express the Baar gene (De Block et al., 1987) that confers resistance to bialophos (Meiji Seika Kaisha Ltd., Tokyo, Japan) and glufosinate ammonium (AgrEvo, Wilmington, Del.). The transgenic overexpression of the Pina-D1a (Pina) and Pinb-D1a (Pinb) coding sequences is under the control of the Glu1Dy10 seed-specific high molecular weight glutenin promoter reported by Blechl and Anderson (1996). The crosses generated two populations in the HL/McN and HL/CR background, segregating for the native Ha locus from HL, CR, or McN and the presence or absence of the transgene (Pina or Pinb). The F₂ generation for each cross was grown in the greenhouse.

To identify F₂ derived F₃ seed pools homozygous for the presence or absence of the transgene, 18 F₂ derived F₃ seeds per line were planted in the greenhouse. Plants were sprayed with 0.1% glufosinate ammonium at the two leaf stage. The plants were scored as being resistant or susceptible after 7 days. Resistant plants stayed green while susceptible plants were killed. The F₂ parent was classified as a Pin transgene homozygous positive or a Pin transgene homozygous negative genotype if >11 consecutive F₃ progeny were glufosinate-resistant or glufosinate-susceptible, respectively. F₃ progeny with mixed herbicide results, most often segregating 3:1 resistant:susceptible, were considered to have a heterozygous-Pin F₂ parent. These lines were excluded from subsequent experiments.

To identify the allelic state of the Ha locus, leaf tissues were taken from F₃ progeny lines arising from a single F₂ parent homozygous positive or negative for the transgene. Young leaf tissues were pooled from >10 individual F₃ plants from a single F₂ parent and genomic DNA extracted according to Riede and Anderson (1996). The Ha locus genotype was determined using cleaved amplified polymorphic marker tests (CAPS) as described in Wanjugi et al. (2007a). Identification of genotypes homozygous for the presence or absence of the transgene and for the segregating native Ha locus resulted in four genotype classes for each cross. We used two of the four genotype classes in our experiment. The two classes were those inheriting the McN Ha locus in the crosses to McN or the CR Ha locus in crosses to CR in combination with the presence or absence of an added transgene. The classes inheriting the native Ha from HL were not included as the effect of PIN overexpression in the presence of the HL locus has been previously reported (Hogg et al. 2004, 2005). For this study, we used a subset of three random lines from the two genotype classes of F₃ derived lines homozygous for the CR or McN Ha locus and the presence of absence of the transgene.

Field Planting and Seed Traits. Genotypes were grown in 2005 in a randomized block design with two replications at the Montana State University-Bozeman Arthur H. Post Field Research Farm as described in Wanjugi et al. 2007a.

Grain hardness was determined using the Single Kernel Characterization System 4100 (SKCS, Perten Instruments, Springfield, Ill.) and grain protein was determined using near-infra red transmission using an infratec 1225 Grain Analyzer (Foss North America Incorporated, Eden Prairie, Minn.) from a subsample of grain from each plot. The genotypes were also analyzed for PIN protein levels and polar lipid content. All analyses were replicated twice.

Extraction of Total and Starch Bound Puroindolines. Extraction and fractionation of total TX-114 soluble puroindoline proteins was done as described by Giroux et al. (2003). Quantification of total TX-114 soluble puroindoline proteins was carried out as described by Wanjugi et al. (2007a). Water washed prime starch used for friabilin (starch bound PIN) extraction was prepared from straight grade flour as described by Wolf, (1964). To obtain straight grade flour, whole grain samples were milled on a Brabender Quadrumat Jr. Mill (Brabender GmbH, Duisburg, Germany) as described by Wanjugi et al. (2007b). Friabilin was extracted by adding 400 μl of 50% isopropanol/0.5M NaCl to 150 mg of water washed starch, vortexed and samples were incubated for 1 h at room temperature. After incubation, the samples were centrifuged at 13000 g for 3 min and supernatant transferred to a new tube. To the supernatant, 520 μl of acetone was added and the samples were incubated overnight at −20° C. After incubation at −20° C., the samples were centrifuged at 13,000 g for 3 min. The supernatant was discarded and the pellet washed once with 500 μl acetone, dried, and suspended in SDS-PAGE sample buffer using 240 μl of sample buffer for each 100 mg starch used minus any reducing agents. Samples were heated at 70° C. for 15 min, and 10 μL (1×) was loaded into 10-20% Tris-HCl, 160 by 160 by 1.5 mm polyacrylamide gels (BioRad, Hercules, Calif.). Total friabilin was quantified using a Heron (soft wheat) scale ranging from 0.5× to 4×, where 1× equals 10 μl.

Immunofluorescent localization. Tissue preparation and fluorescent staining were carried out according to the methods of Dubreil et al. (1998) with some modifications. Three randomly selected mature wheat seeds from each genotype class and parental controls described above were fixed in a 4% paraformaldehyde, 100 mM phosphate buffer, pH 7, for two days. After rinsing twice in a 100 mM phosphate buffer, pH 7, the seeds were tangentially cut into 2 mm² pieces and dehydrated with 70, 85, 100% (v/v) ethanol-water mixtures for two hours each. Samples were placed in 30, 40, 50, 60, 70, 80, 90% (v/v) mixtures of medium grade London Resin white (Sigma Aldrich, Oakville, Canada)-ethanol for two hours each. Seeds were infiltrated three times in 100% London Resin white (for 12 hours each, followed by polymerization at 60° C. for 48 hours. 2 μm seed sections were cut with a Diatome MT485 diamond knife on a LKB Ultratome III type 8801A ultra microtome (Pharmacia-LKB, Sweden), collected in a glass well, and heated mildly to adhere the sample to a glass slide. Seed sections were rinsed in phosphate buffered saline (PBS) for 20 minutes and blocked with 2% goat serum-PBS for one hour at room temperature. Three ten-minute PBS washes were performed on the sections, followed by a one hour staining with either rabbit anti-PINA or anti-PINB antisera in 2% goat serum-PBS. Sections were washed in PBS three times for ten minutes each. Anti-PINA and anti-PINB antisera were diluted 1/400 in 2% goat serum-PBS. Anti-PINA and anti-PINB antisera (Dubreil et al., 1998) treated sections were incubated with a 1/400 dilution of goat anti-rabbit Alexa Fluor 488 conjugated antibody (Invitrogen, Burlington, Ontario) in 2% goat serum-PBS. Sections were washed three times for 10 minutes each in PBS. A drop of ProLong® Gold Antifade (Invitrogen, Burlington, Ontario) was applied to each section to prevent photo bleaching before adding cover slips. Micrographs were obtained from an Axiophot microscope (Zeiss, Oberkochen, Germany) equipped with a Hamamatsu C5985-02 camera (Hamamatsu, Hamamatsu, Japan). Exposure times for phase contrast (0.1 s) and fluorescent (1 s) micrographs were kept constant for all seed samples. Fluorescent and phase contrast image overlays were performed with Metamorph® Imaging System software (Molecular Devices, Sunnyvale, Calif.), using arithmetic “add” function.

Extraction, Chromatography and Quantification of Lipid Fractions. Whole wheat flour was prepared from seeds ground on UDY mill fitted with a 0.5 mm screen (UDY Co., Fort Collins, Co) while water washed prime starch was isolated from Brabender Quadrumat Jr. Mill straight-grade flour as described by Wolf (1964). Extraction of bound polar lipids from whole wheat flour and prime starch and group separation of bound polar lipids and thin layer chromatography (TLC) of polar lipid fractions was performed as described by Morrison et al. (1980) and Greenblatt et al. (1995). Free lipids were first extracted from flour and starch with hexane (sample to hexane ratio of 1:10) for 0.5 h and discarded. Bound lipids were then extracted sequentially from flour and starch with propan-2-ol and water (90:10) at 1:6 and 1:3 sample-to-solvent ratio respectively to ensure that extraction went to completion. Group separation of extractable polar lipids was accomplished as described by Greenblatt et al. (1995). Bound polar lipids extracts from each line in a genotype class possessing either the McN Ha locus or CR Ha with or without the added transgene were spotted in triplicate on glass plates (20×20 cm) coated with a 0.3 mm layer of Silica Gel (Whatman, Maidstone, UK). TLC plates were then developed with the appropriate solvent, visualized (Greenblatt et al., 1995) and quantified. Phospholipids (PL) and glycolipids (GL) were identified by comparing their R_{F} values (ratio of the distance traveled by a lipid species compared to the solvent) with commercially available glycolipid and phospholipid standards obtained from Sigma (Sigma Chemical Co, St. Louis, Mo.). Twenty five mg of each phospholipid standard was dissolved in 2.5 ml chloroform while 1 mg of each glycolipid standard was dissolved in 50 ml chloroform. A loading standard curve of lipid standards with known concentration on a TLC plate was used to quantify the amount of PL and GL from prime starch and flour samples. Data from each lipid species were analysed via analysis of variance where the model included independent replication genotype class and random lines within genotype classes using PROC GLM in SAS (SAS Institute, Inc, 2004). Data were deleted from the analysis when 0 (non-detectable) was obtained for a lipid species. Least significant difference (LSD) was used to compare genotype class means.

Results. Grain hardness and grain protein content were analyzed from a subset of three lines randomly selected from each of the two genotype classes, homozygous for CR or McN Ha locus and presence or absence of the added transgene. Genotypes lacking the added transgene did not substantially vary in grain hardness and grain protein in either the CR/HL or the HL/McN cross (Table VI).

TABLE VI
Mean grain hardness and grain protein content for a subset of F₃-derived
progeny derived from crosses of either ‘Canadian Red’ (Pina-D1a/Pinb-D1e) or ‘McNeal’
(Pina-D1b/Pinb-D1a) with ‘Hi-Line’ (Pina-D1a/Pinb-D1b) derived transgenic
isolines with added Pina (HGA3) or Pinb (HGB12). Presence or absence
of transgene is denoted as + or −, respectively. F₃ line values are means of
three random lines from each genotype class with standard errors presented in ( ).

| | | | | | Grain |
|---|---|---|---|---|---|
| Parental | | Transgenic | | Grain | Protein |
| Varieties^{a} | Ha^{b} | Parent^{c} | Transgene | Hardness^{d} | (g/kg)^{e)} |
| Heron | Pina-D1a/Pinb-D1a | None | None | 30.4( ) | 133( ) |
| Canadian Red | Pina-D1a/Pinb-D1e | None | None | 71.6(2.12) | 136(0.81) |
| McNeal | Pina-D1b/Pinb-D1a | None | None | 95.1(2.14) | 149(3.06) |
| HiLine | Pina-D1a/Pinb-D1b | None | None | 78.9(1.63) | 152(3.37)) |
| Null parent | F₃ lines homozygous for presence or absence of Pin transgene and Ha locus) | | | | |
| Canadian Red | Pina-D1a/Pinb-D1e | HGA3+ | + | 43.5(1.37) | 146(0.16) |
| | | HGA3− | − | 68.5(2.34) | 148(0.23) |
| | | HGB12+ | + | 15.9(1.72) | 157(0.11) |
| | | HGB12− | − | 70.3(2.42) | 152(0.23) |
| McNeal | Pina-D1b/Pinb-D1a | HGA3+ | + | 28.2(1.92) | 157(0.14) |
| | | HGA3− | − | 88.4(2.16) | 157(0.21) |
| | | HGB12+ | + | 45.5(1.72) | 157(0.17) |
| | | HGB12− | − | 84.5(2.10) | 158(0.20) |

^{a}Hi-Line, Canadian Red, McNeal and Heron are hard wheat non-transgenic controls. Heron and non-transgenic Hi-Line and are listed for comparative purposes only and was not used in any cross.
^{b}Puroindoline genes present at the Ha locus. The Pina-D1a and Pinb-D1a alleles produce functional PINA and PINB, respectively. The Pina-D1b and Pinb-D1e alleles are null alleles for PINA and PINB, respectively. The Pinb-D1b allele produces a non-functional PINB.
^{c}Hi-Line derived transgenic lines contain either Pina or Pinb transgene. Recombinant progeny lines are homozygous for CR Ha locus (Pina-D1a/Pinb-D1e) or McN Ha locus (Pina-D1b/Pinb-D1a) with (+) or lacking (−) added transgene.
^{d}Determined by single kernel characterization system (SKCS).
^{e}Determined by Infratec 1225 grain analyzer

Addition of PINB to McN Ha locus genotypes and addition of PINA to CR Ha locus genotypes resulted in intermediate grain hardness with a mean value of 45.5 and 43.5 respectively (Table VI). Addition of PINB to CR Ha locus genotypes resulted in much softer texture (grain hardness mean value=15.9) than the addition of PINA to McN Ha locus genotypes (grain hardness mean value=28.2). Grain protein did not vary substantially with the addition of either PIN to either McN or CR Ha locus genotypes.

Total TX-114 extractable and prime starch-associated proteins levels, were extracted, fractionated using SDS-PAGE, and visualized by direct staining. The amounts of PINA and PINB present in ‘Heron’ (soft wheat) were used as a reference to which other samples were compared. The amount of total TX-114 extractable PINA in CR was comparable to ‘Heron’ though no PINB was present in CR (Table VII).

TABLE VII
Puroindoline levels quantified from TX-114 and friabilin extracts for parental controls and F₃ lines derived from
crosses of either ‘Canadian Red’ (Pina-D1b/Pinb-D1e) or ‘McNeal’ (Pina-D1b/Pinb-D1a) with ‘Hi-Line’
(Pina-D1a/Pinb-D1b) transgenic isolines with added Pina (HGA3) or Pinb (HGB12). F₃ line values are means of
three random lines from each genotype class with standard errors presented in ( ).

| | | Transgenic | | TX-114 Protein ^{a} | | | Friabilin ^{b} | | Total Starch- |
|---|---|---|---|---|---|---|---|---|---|
| Null Parent | Ha | Parent | Transgene | PINA | PINB | Total PIN | PINA | PINB | Associated PIN |
| Heron | Pina-D1a/Pinb-D1a | None | None | 1.00 | 1.00 | 2.00 | 1.00 | 1.00 | 2.00 |
| Canadian red (CR) | Pina-D1a/Pinb-D1e | None | None | 1.10 | 0.00 | 1.10 | ND | 0.00 | 0.00 |
| McNeal (McN) | Pina-D1b/Pinb-D1a | None | None | 0.00 | ND ^{c} | 0.00 | 0.00 | ND | 0.00 |
| Canadian red | Pina-D1a/Pinb-D1e | HGA3 | + | 4.10 | 0.00 | 4.10 | 1.58 | 0.00 | 1.58 |
| | | HGB12 | + | 1.00 | 2.80 | 3.80 | 0.71 | 2.54 | 3.25 |
| McNeal | Pina-D1b/Pinb-D1a | HGA3 | + | 4.30 | 0.90 | 5.20 | 2.20 | 0.92 | 3.12 |
| | | HGB12 | + | 0.00 | 3.00 | 3.00 | 0.00 | 2.47 | 2.47 |

^{a} Means of three randomly selected lines homozygous for the transgene and the Ha locus. Values are relative levels of TX-114 extractable PIN proteins compared to Heron (Soft) scale of 1× to 8× where 1× = 6 μl (240 μl SDS-PAGE sample buffer/100 mg ground seeds)
^{b} Starch associated PINA and PINB (friabilin) levels from water washed starch. Values are relative levels of starch associated PIN proteins compared to Heron (Soft) scale of 1× to 5× where 1× = 10 μl (240 μl SDS-PAGE sample buffer/100 mg starch).
^{c} ND (non-detectable, <5% of Heron control value, with method used)

McN had no PINA present and almost no detectable PINB. Neither CR nor McN accumulated starch bound puroindoline (friabilin) (Table VII). The amount of TX-114 extractable puroindoline in PIN null genotypes with added PINA or PINB was comparable to that observed by Wanjugi et al. (2007a). Addition of PINA to McN and CR Ha locus genotypes resulted in higher levels of total TX-114 extractable PIN than addition of PINB to CR and McN Ha locus genotypes (Table VII). In contrast, genotypes with the CR Ha locus and added PINB had greater starch-bound PIN levels than the McN Ha genotypes with added PINA. Most of the added PINB in both McN CR Ha locus genotypes was associated with prime starch as opposed to PINA in which a smaller portion of TX-114 extractable protein was present on starch (Table VII).

Immunofluorescent localization of PINA and PINB. To accurately localize PINs in the wheat endosperm, the specificity and cross-reactivity of the anti-PINA and anti-PINB antisera were tested. CR Ha locus genotypes in the absence of a transgene or CR Ha locus genotypes with added PINA did not react with the anti-PINB antibody (data not provided). Similarly, McN Ha locus genotypes in the absence of a transgene or McN Ha locus genotypes with added PINB did not react with the anti-PINA antibody (data not provided). These observations confined the absence of native PINA in McN Ha and PINB in CR Ha locus genotypes. We assessed the localization of PINA and PINB in the presence or absence of the other protein on starch granules using dry sections of mature seeds which were either expressing native Ha or transgenically overexpressed PINA or PINB. In all cases, PINs localized entirely on the surface of starch granules with little to no non-localized fluorescence (data not provided). The results obtained with the anti-PINA antibody demonstrated that PINA localization is the same in the presence or absence of PINB (data not provided). Similarly, the use of the anti-PINB antibody demonstrated that PINB binds exclusively to the surface of starch in the presence or absence of PINA (data not provided).

Polar Lipids Extractable from Starch or Starch Polar Lipid Content. The effect of Pin addition upon polar lipids present in whole wheat flour and water washed prime starch was determined (Table VIII).

TABLE VIII
Composition of polar bound lipids from whole wheat flour (μg lipid/1 g of flour) for parental varieties and F₃ lines derived
from crosses of either ‘Canadian Red’ (Pina-D1b/Pinb-D1e) or ‘McNeal’ (Pina-D1b/Pinb-D1a) with ‘Hi-Line’ (Pina-D1a/Pinb-D1b) transgenic isolines
with added Pina (HGA3) or Pinb (HGB12). F₃ line values are means of three random lines from each genotype class with standard errors in ( ).

| | | | (μg/1 g of whole wheat flour) | | | | | | | |
|---|---|---|---|---|---|---|---|---|---|---|
| Parental | | | Transgenic | Glycolipids^{a} | | | | Phospholipids^{b} | | |
| varieties | Ha | Transgene | Parent | DGDG | MGDG | Total GL | NAPE | DOPC | LPC | Total PL |
| Heron | Pina-D1a/ | None | None | 90.10(5.77) | 33.33(1.67) | 123.43(17.12) | 19.00(1.47) | 4.12(1.39) | 2.00(0.67) | 23.13(4.82) |
| | Pinb-D1a | | | | | | | | | |
| Canadian | Pina-D1a/ | None | None | 65.23(2.88) | 21.00(1.57) | 86.23(9.42) | 10.67(0.67) | 2.50(0.29) | 5.80(0.83) | 18.97(2.84) |
| Red | Pinb-D1e | | | | | | | | | |
| McNeal | Pina-D1b/ | None | None | 48.33(4.40) | 21.75(3.94) | 70.08(9.61) | 19.16(0.84) | 1.96(0.84) | 4.67(1.83) | 25.79(3.56) |
| | Pinb-D1a | | | | | | | | | |
| Hi-Line | Pina-D1a/ | None | None | 45.00(5.00) | 16.33(1.85) | 61.33(7.27) | 18.60(1.87) | 3.83(0.44) | 6.0(0.57) | 28.43(2.76) |
| | Pinb-D1b | | | | | | | | | |
| Null | | | | | | | | | | |
| parent | | | F₃ lines homozygous for presence or absence of Pin transgene and Ha locus | | | | | | | |
| Canadian | Pina-D1a/ | + | HGA+ | 82.33.(5.14) | 20.83(1.60) | 103.16(9.67) | 25.33(1.67) | 3.08(0.39) | 2.83(0.46) | 31.24(2.61) |
| Red | Pinb-D1e | − | HGA− | 57.67(6.43) | 14.33(1.17) | 72.00(7.24) | 26.33(2.84) | 2.67(0.54) | 3.50(0.57) | 38.67(2.82) |
| | | + | HGB+ | 110.00(8.16) | 24.01(3.96) | 134.01(13.68) | 21.83(0.74) | 3.00(0.22) | 3.41(0.52) | 28.24(2.15) |
| | | − | HGB− | 47.00(2.23) | 12.33(1.31) | 59.33(5.37) | 24.00(0.96) | 2.71(0.20) | 3.10(0.16) | 38.31(2.43) |
| McNeal | Pina-D1b/ | + | HGA+ | 100.00(5.42) | 31.50(4.73) | 131.5(11.72) | 21.67(0.56) | 4.67(0.80) | 9.50(0.87) | 35.84(2.43) |
| | Pinb-D1a | − | HGA− | 48.33(5.98) | 18.83(2.07) | 67.16(9.05) | 28.33(3.57) | 2.08(0.15) | 3.58(0.52) | 33.99(2.81) |
| | | + | HGB+ | 81.67(7.03) | 27.50(4.20) | 109.17(9.05) | 22.83(1.86) | 6.75(0.53) | 9.33(0.60) | 38.75(1.74) |
| | | − | HGB− | 39.67(3.48) | 14.17(1.07) | 53.84(4.21) | 24.17(2.38) | 1.13(0.43) | 3.33(0.36) | 28.63(2.61) |
| | | | LSD(0.05)^{c} | 22.1 | 4.2 | 19.7 | 5.3 | 1.3 | 1.3 | 8.2 |

^{a}Quantified values for whole wheat flour glycolipids, DGDG (digalactosyldiglyceride), MGDG (monogalactosyldiglyceride). Values are means of three randomly selected lines per genotype class homozygous for the transgene and Ha; replicated twice.
^{b}Quantified values for whole wheat flour phospholipids. NAPE (N-acyl phosphatidylethalonolamine), PC, (Phospatidylcholine), NAPC (N-acyl Phospatidylcholine). Values are means of three randomly selected lines per genotype class homozygous for the transgene and Ha; replicated twice
^{c}LSD values compares line means within a genotype class.

Glycolipids were abundant and mainly composed of DGDG which was 2-3 times higher than MGDG in whole wheat flour of all genotypes. Phospholipids were less (about 25% of the total polar lipids content) and mostly composed of NAPE. Whole wheat flour of the hard wheats parental varieties, CR, McN and HL possessed lower amounts of glycolipids than the soft wheat Heron, but similar levels of phospholipids (Table VIII). The same trend was observed among hard textured progeny lines lacking the transgene. Whole wheat flour from transgenic soft genotypes had increased levels of bound polar lipids compared to intermediate or hard textured genotypes without the transgene (Table VIII). In this respect, glycolipids were greatest for the soft genotypes (CR Ha locus with added PINB and McN Ha locus with added PINA) followed by intermediate textured genotypes (CR Ha locus with added PINA and McN Ha locus with added PINB), and least for hard genotypes (CR Ha and McN Ha without transgene). In contrast, the phospholipid content of flour of McN Ha and CR Ha locus genotypes with or lacking the transgene, remained relatively equivalent (Table VIII). However, in soft wheat flours there was an increase in total bound glycolipid content, with decreased grain hardness (Table VI and Table VIII).

To test if PIN expression affects the amount of starch polar lipids, we prepared water washed prime starch from straight-grade flour by standard methods (Wolf, 1964). This allowed us to determine extractable residual polar lipids on the surface of starch and eliminate contamination with fractions originating from the germ, bran, and aleurone layer. Starch polar lipids were much reduced in water washed prime starch compared to whole wheat flour with extractable glycolipids and phospholipids being essentially non detectable when starches from hard wheats were analyzed (Table IX).

TABLE IX
Composition of polar bound lipids from water washed starch (μg lipid/1 g of flour) for parental controls
and F₃ lines derived lines from crosses of either ‘Canadian Red’ (Pina-D1b/Pinb-D1e) or ‘McNeal’ (Pina-D1b/Pinb-D1a)
with ‘Hi-Line’ (Pina-D1a/Pinb-D1b) transgenic isolines with added Pina (HGA3) or Pinb (HGB12). F₃ line F₃ line
values are means of three random lines from each genotype class with standard errors in ( ).

| | | | (μg/1 g of prime starch) | | | | | | | |
|---|---|---|---|---|---|---|---|---|---|---|
| | | | Transgenic | Glycolipids^{a} | | | Phospholipids^{b} | | | |
| Genotype | Ha | Transgene | parent | DGDG | MGDG | Total GL | NAPE | DOPC | LPC | Total PL |
| Heron | Pina-D1a/ | None | None | 34.42(2.88) | 3.33(0.89) | 37.33(8.87) | 7.50(1.44) | ND^{a} | ND | 7.50(1.44) |
| | Pinb-D1a | | | | | | | | | |
| Canadian | Pina-D1a/ | None | None | ND | ND | ND | ND | ND | ND | ND |
| red | Pinb-D1e | | | | | | | | | |
| McNeal | Pina-D1b/ | None | None | ND | ND | ND | ND | ND | ND | ND |
| | Pinb-D1a | | | | | | | | | |
| HiLine | Pina-D1a/ | None | None | ND | ND | ND | ND | ND | ND | ND |
| | Pinb-D1b | | | | | | | | | |
| Null parent | | | F₃ lines homozygous for presence or absence of Pin transgene and Ha locus | | | | | | | |
| Canadian | Pina-D1a/ | + | HGA+ | 32.50(2.81 | 4.00(0.63) | 37.00(4.51) | 8.67(1.28) | ND | ND | 8.67(1.28) |
| Red | Pinb-D1e | − | HGA− | ND | ND | ND | ND | ND | ND | ND |
| | | + | HGB+ | 78.33(3.07) | 13.41(1.49) | 86.45(9.92) | 12.50(1.8) | ND | ND | 12.50(1.8) |
| | | − | HGB− | ND | ND | ND | ND | ND | ND | ND |
| McNeal | Pina-D1b/ | + | HGA+ | 33.33(4.22) | 4.67(0.80) | 38.16(4.78) | 12.08(1.00) | ND | ND | 12.08(1.00) |
| | Pinb-D1a | − | HGA− | ND | ND | ND | ND | ND | ND | ND |
| | | + | HGB+ | 45.00(5.47) | 10.5(2.01) | 54.72(5.89) | 10.83(1.17) | ND | ND | 10.83(1.17) |
| | | − | HGB− | ND | ND | ND | ND | ND | ND | ND |
| | | | LSD(0.05)^{c} | 12.3 | 2.8 | 12.4 | 3.5 | | | 3.5 |

^{a}Quantified values for water washed prime starch glycolipids, DGDG (digalactosyldiglyceride), MGDG (monogalactosyldiglyceride). Values are means of three randomly selected lines per genotype class homozygous for the transgene (+/−) and Ha; replicated twice.
^{b}Quantified values for water washed prime starch phospholipids. NAPE (N-acyl phosphatidylethalonolamine), PC, (Phospatidylcholine). NAPC (N-acyl Phospatidylcholine). Values are means of three randomly selected lines per genotype class homozygous for the transgene (+/−) and Ha; replicated twice.
^{c}ND (non-detectable, <5% of Heron control value, with method used)
^{d}LSD values compares line means within a genotype class.

The presence of glycolipids (DGDG, MGDG) from starch of wild type soft (Heron) and upon the addition of either Pin to CR Ha or McN Ha locus genotypes, followed the same pattern to that of whole wheat flour (Table VIII and IX). However, glycolipids from water washed starch were more prevalent in CR Ha and McN Ha locus genotypes containing added PINB than II genotypes containing added PINA (Table IX). Phospholipids from water washed starch were mainly composed of NAPE, with transgenic soft genotypes having higher levels respectively (Table IX). In all cases, our results concurred with previous findings that, DGDG>MGDG and GL>PL in whole wheat flour and water washed prime starch (Morrison et al., 1980; Morrison and Hargin, 1980).

Discussion. Soft genotypes (Table VI) had increased TX-114 total PIN (Table VII) and increased amounts of PIN on the surface of starch granules. Addition of PINB to CR Ha locus genotypes led to more starch associated PIN than addition of PINA to McN Hal locus genotypes (Table VII). Likewise, more PINB associated with starch upon addition of PINB to McN Ha locus than addition of PINA to CR Ha locus genotypes (Table VII). Increased association of PINB to starch was also confined by abundant immunofluorescence upon addition of PINB to McN Ha and to CR Ha locus genotypes.

Whole wheat flour contained more bound polar lipids than water washed starch (Table VIII and IX). This is due to the occurrence of varying amounts of polar lipids in various kernel fractions such as bran, aleurone and the germ which are absent in starch. Higher contents of glycolipids especially digalactosydiglycerides (DGDG) were consistent in both flour and starch, with addition of PINB to CR Ha locus resulting in the highest glycolipids content. The principle phospholipids in flour were negatively charged N-acyphospatidylethialonamines (NAPE) and neutral charged phosphatidycholines (LPC and PC) but only NAPEs were present in water washed starch of soft and non detectable in hard genotypes. Among polar lipids, GL are related to end product quality in hard wheat with increased GL content associated with improved milling, dough mixing, and bread making quality attributes.

Glycolipids (DGDG) and phospholipids (NAPE) residual membrane polar lipids predominate on the surface of starch, and their abundance corresponds with increased starch bound PINs (friabilin) and grain softness. This is however in contrast with Konopoka et al. (2005) who indicated that a higher content of DGDG corresponds to increased grain hardness. Because puroindolines are positively charged (Blochet et al., 1993), the main electrostatic interaction of PINs with polar lipids on the surface of starch mostly involves DGDGs, MGDGs and negatively charged NAPEs, and that these lipid fractions can be used as a biochemical marker for grain hardness or could explain variation in baking and milling traits observed between hard and soft wheats.

Our results show that PINB binds more to GL than PINA on starch; however, both PINs increase the abundance of GL in flour of soft relative to hard genotypes, with this effect, increasing greater in transgenic soft genotypes relative to wild type soft (‘Heron’). Two extra tryptophan residues outside the tryptophan-rich domain in PINB likely enhance polar lipid affinity of the protein and therefore increasing lipid-PINB interaction on the surface of starch. Increased friabilin and bound polar lipids upon addition of PINB to CR Ha locus would also suggest that increasing PINB enhances PIN association to polar lipids and starch binding.

These studies show that PIN expression is strongly associated with GL and PL lipid levels in whole seed meal and starch. Certainly, there are several distinct possible explanations for the linkage between PIN expression and polar lipid content. The first is that PINs regulate the expression of polar lipids. This seems quite unlikely as PINs are storage proteins with no known or predicted enzymatic activity. The second possibility is that the Ha locus contains gene(s) linked to Pins that regulate polar lipid content. This too seems unlikely as the Ha locus has been cloned from numerous Triticeae sp and only the Pin, Gsp (Grain softness protein), and several pseudogenes lie within close proximity (Chantret et al. 2005). Linked polar lipid biosynthetic genes also seem unlikely as our transgenic lines in which only Pin expression is altered contain large increases in polar lipids. The third and most likely possibility is that active PINs stabilize polar lipids on the surface of starch granule membranes preventing breakdown during seed dry down and maturation. Alternatively, the low levels of GL and PL in hard and high levels in soft wheat flour and starch leaves the intriguing possibility that starch bound polar lipids are formed during later stages of kernel maturation when PIN expression is highest in soft (Hogg et al., 2004) or lipid degradation occurs during kernel ripening and maturation in hard wheats.

These studies demonstrate that puroindoline expression is associated with increased bound polar lipids on starch. Glycolipids and phospholipids (DGDG and NAPE) contribute most to physiochemical interaction between PINA and PINB on the surface of starch that influence endosperm texture. It is also apparent that PINB binds polar lipids (glycolipids) on the surface of starch with higher affinity than PINA but, abundant polar lipid content is associated with the presence of both PINA and PINB and soft endosperm texture. A possible explanation for these results is that active PINs stabilize polar lipids on the surface of starch granule membranes preventing breakdown during kernel ripening and maturation. These results provide further evidence to support the hypothesis that the association of friabilin components (PINA and PINB) with starch granule surface is mediated by membrane bound polar lipids that affect endosperm texture and that PINs do not directly bind to starch.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES FOR WHICH A COMPLETE CITATION MAY NOT BE PROVIDED IN THE TEXT OF THE SPECIFICATION

The full contents of each of the following listed documents are herein incorporated by reference in their entireties.
Agri. Research Station, Maruteru. 1986. In Sixty years of rice research at Maruteru. 73 pages. Anon. 1988. Sheath blight: Losses cost $67 million a year. Rice J. 41:5-8.
Asuyama, H. 1965. Morphology, taxonomy, host range and life cycle of Pyricularia oryzae. pp. 9-22. In The Rice Blast Disease. Proc. Symp. IRRI. John Hopkins University Press, Baltimore, Md.
Awoderu, V. A. 1990. Yield loss attributable to neck-rot of rice caused by Pyricularia oryzae, Cav. In Dote d'lvoire. Tropical Pest Management 36:394-396.
American Association of Cereal Chemists. 2003. Approved Methods of the American Association of Cereal Chemists. 10th ed. AACC.
Anjum, F. M., and Walker, C. E. Review on the significance of starch and protein to wheat kernel hardness. 1991. Journal of the Science of Food and Agriculture. 56: 1-13.
Baker, R. J. 1977. Inheritance of kernel hardness in spring wheat. Crop Sci. 14:960-962.
Blochet, J. E., Chevalier, C., Forest, E., Pebay-Peyroula, E., Gautier, M. F., Joudrier, P., Pezolet, M., and Marion, D. 1993. Complete amino acid sequence of puroindoline, a new basic and cystine-rich protein with a unique tryptophan-rich domain, isolated from wheat endosperm by Triton X-114 phase partitioning. FEBS 329:336-340.
Bordier, C. 1981. Phase separation of integral membrane proteins in Triton X-114 solution. J. Biological Chemistry 256:1604-1607.
Broekaert, W. F., Cammue, B. P. A., De Bolle, M. F. C., Thevissen K., De Samblanx, G. W., and Osborn, R. W. 1997. Antimicrobial peptides from plants. Crit. Rev. Plant Sci. 16:297-323.
Beecher, B. A., Smidansky, B. E., and Giroux, M. J. 2002. Expression of wild-type Pinb sequence in transgenic wheat complements a hard phenotype. Theor. Appl. Genet. 105:870-877.
Bergthaller, W. and Lindhauer, M. G. 2004. Proposal for rapid evaluation of the separation potential of concentrated wheat flour/water slurries. Detmold, Mathias Look, Ibbenburen Getresdetechnologie (Cereal Technology) J8, 239-245.
Blechl, A. E., and O. D. Anderson. 1996. Expression of a novel high-molecular-weight glutenin subunit gene in transgenic wheat. Nat Biotech 14:875-879.
Barlow, K. K., Buttrose, M. M., Simmonds, D. H., and Vesk, M. 1973. The nature of the starch-protein interface in wheat endosperm. Cereal Chem. 50, 443-454.
Carmona, -M. J.; Molina, -A.; Fernandez, -J. A.; Lopez-Fando, -J. J.; Garcia-Olmedo, -F. 1993. Expression of the alpha-thionin gene from barley in tobacco confers enhanced resistance to bacterial pathogens. Plant-J. 3 (3):457-462.
Christensen, A. H. and Quail, P. 1996. Ubiquitin promoter-based vectors for high-level expression of selectable and/or screenable marker genes in monocotyledonous plants. Transgenic Res. 5:213-218.
Campbell, J. B., Martin, J. M., Crutcher, F., Meyer, F. D., Clark, D. R., and Giroux, M. J. 2007. Effects on soft wheat (Triticum aestivum L.) quality of increased puroindoline dosage. Cereal Chem. 84:80-87.
Cane, K., Spackman, M., and Eagles, H. A. 2004. Puroindoline genes and their effects on grain quality traits in southern Australian wheat cultivars. Aust. J. Agric. Res. 55:89-95.
Chen, F., He, Z. H., Xia, X. C., Xia, L. Q., Zhang, X. Y., Lillemo, M., and Morris, C. F. 2006. Molecular and biochemical characterization of puroindoline a and b alleles in Chinese landraces and historical cultivars. Theor. Appl. Genet. 112:400-409.
Czuchajowska, Z., and Pomeranz, Y. 1993. Protein concentration and prime starch from wheat flours. Cereal Chem. 70:701-706.
Caldwell. K. S., P. Langridge, and W. Powell. 2004. Comparative sequence analysis of the region harboring the hardness locus in barley and its collinear region in rice. Plant Phys. 136:3177-3190.
Capparelli, R., Amaroso. M. A, Palumbo. D, Iannacoone. M, Faleri. C, and M. Cresti. 2005. Two plant puroindolines colocalize in wheat seed and in vitro synergistically fight against pathogens. Plant Mol. Biol. 58:857-867.
Chantret, N. J. Salse, F. Sabot, S. Rahman, A. Bellec, B Laubin, I. Dubois, C. Dossat, P. Sourdille, P. Joudrier, M. F. Gautier, L. Cattolico, M. Beckert, S. Aubourg, J. Weissenbach, M. Caboche, M. Bernard, P. Leroy, and B. Chalhoub. 2005. Molecular basis of evolutionary events that shaped the Hardness locus in diploid and polyploidy wheat species Triticum and Aegilops. Plant Cell. 17:1033-1045.
Clark, J. A., H. H. Love, E. F. Gaines. 1926. Registration of standard wheat varieties. J. Amer. Soc. Agron. 18 (10):920-922.
Dale, P. J., Irwin, J. A. and Scheffler, J. A. 1993. The experimental and commercial release of transgenic crop plants. Plant Breeding. 111: 1-22.
Disthaporn, S. 1993. Current rice blast epidemics and their management in Thailand. International symposium on Rice Blast Disease. Univ. of Wisconsin, Madison (abstract).
Dubreil, L., Gaborit, T., Bouchet, B., Gallant, D. J., Broekaert, W. F., Quillien, L., and Marion, D. 1998. Spatial and temporal distribution of the major isoforms of puroindolines (puroindoline-a and puroindoline-b) and non specific lipid transfer protein (ns-LTPel) of Triticum aestivum seeds. Relationships with their in vitro antifungal properties. Plant Science 138: 121-135.
De Block, M., J. Botterman, M. Vandewiele, J. Dockx, C. Thoen, V. Gossele, N. Rao Movva, C. Thompson, M. Van Montagu, and J. Leemans. 1987. Engineering herbicide resistance in plants by expression of a detoxifying enzyme. EMBO J. 6:2513-2518.
De La Roche, and C. J. Andrews. 1973. Changes in phospholipid composition of a winter wheat cultivar during germination at 2 C and 24 C. Plant Physiol. 51:468-473.
Dubreil, L., Meliande, S, Chiron, H, Compoint, J.-P, Quillen, L, Branlard, G, and Marion, D. 1998. Effect of puroindolines on the bread making properties of wheat flour. Cereal Chem. 75, 222-229.
Dubreil, L., J. Compoint, and D. Marion. 1997. Interaction of puroindoline with wheat flour polar lipids determines foaming properties. J. Agric. Food Chem. 45: 108-116.
Epple, P., Apel, K., and Bohlmann, H.1997. Overexpression of an endogenous thionin enhances resistance of Arabidopsis against Fusarium oxysporum. The Plant Cell 9:509-520.
E. G. Heyne. 1959. Registration of improved wheat varieties, XXIII. Agron J 51(11):689.
Fomba, S, N., and Taylor, D. R. 1994. Rice blast in west Africa: Its nature and control. pp. 343-355. In Rice Blast Disease.
Garcia-Olmedo, F., Molina, A., Segura, A., and Moreno, M. 1995. The defensive role of nonspecific lipid transfer proteins in plants. Trends Microbiol. 3:72-74.
Garnier, J., Gibrat, J. F., and Robson, B. 1996. GOR method for predicting protein secondary structure from amino acid sequence. Meth. Enzmol. 266:540-553.
Gatineau, E., Toma, R., Montenay-Garestier, Th., Takechi, M., Fromageot, P., and Menez, A. 1987. Role of tyrosine and tryptophan residues in the structure-activity relationships of a cardiotoxin from Naja nigricollis venom. Biochemist 26:8046-8055.
Gautier, M. F., Aleman, M. E., Guirao, A., Marion, D., and Joudier, P. 1994. Triticum aestivum puroindolines, two basic cystine-rich seed proteins: cDNA analysis and developmental gene expression. Plant Molec. Biol. 25:43-57.
Giroux, M. J., Martin, J. M., Habernicht, D. K., Lanning, S., Hemphill, A., and Talbert, L. (2000) Association of Puroindoline Sequence Type and Grain Hardness in Hard Red Spring Wheat. Crop Science, in press, March April 2000 issue.
Giroux, M. J., and Morris, C. F. 1997. A glycine to serine change in puroindoline b is associated with wheat grain hardness and low levels of starch-surface friabilin. Theor. Appl. Genet. 95: 857-864.
Giroux, M. J., and Morris, C. F. 1998. Wheat grain hardness results from highly conserved mutations in the friabilin components puroindoline a and b. Proc. Natl. Acad. Sci. 95: 6262-6266.
Greenblatt, G. A., Bettge, A. D., and Morris, C. F. 1995. The relationship among endosperm texture, friabilin occurrence, and bound polar lipids on wheat starch. Cereal Chem. 72:172-176.
Greenwell, P., and Schofield, J. D. 1986. A starch granule protein associated with endosperm softness in wheat. Cereal Chem. 63:379-380.
Giroux, M. J., Talbert, L., Habernicht, D. K., Lanning, S., Hemphill, A., and Martin, J. M. 2000. Association of puroindoline sequence type and grain hardness in hard red spring wheat. Crop Sci. 40:370-374.
Greffeuille, V., Abecassis, J., Rousset, M., Oury, F. X., Faye, A., L'helgouac'h, C. B., and Lullien-Pellerin, V. 2006. Grain characterization and milling behavior of near-isogenic lines differing by hardness. Theor. Appl. Genet. 114:1-12.
Giroux, M. J., T. Sripo, S. Gerhardt, and J. E. Sherwood. 2003. Puroindolines: their role in grain hardness and plant defense. Biotechnol. Genet. Eng. Rev. 20:227-290.
Graybosch, R., C. F. Peterson, K. J Moore, J, M. Stems, and D. L. Grant. 1993. Comparative effects of wheat flour protein, lipid, and pentosan composition in relation to baking and milling quality. Cereal Chem. 70:95-101.
Greenwell, P., and J. D. Schofield. 1986. A starch granule protein associated with endosperm softness in wheat. Cereal Chem. 63, 379-380.
Hill, C. P., Yee, J., Selsted, M. E., and Eisenberg, D. 1991. Crystal structure of defensin HNP-3, an amphiphilic dimer: mechanisms of membrane permeabilization. Science 251:1481-1485.
Hori, M. 1969. On forecasting the damage due to sheath blight o rice plants and the critical point for judging the necessity of chemical control of the disease. Review of Plant protection Research. 2:70-73.
Hogg, A. C., Beecher, B., Martin, J. M., Meyer, F. D., Talbert, L. E., Lanning, S., and Giroux, M. J. 2005. Hard wheat milling and bread baking traits affected by the seed-specific overexpression of puroindolines. Crop Sci. 45:871-878.
Hogg, A. C., Sripo, T., Beecher, B., Martin, J. M., and Giroux, M. J., 2004. Wheat puroindolines interact to form friabilin and control wheat grain hardness. Theor. Appl. Genet. 108:1089-97.
Hargin, K. D, and W. R. Morrison. 1980. The distribution of acyl lipids in the germ, aleurone, starch, and non-starch endosperm of four wheat varieties. J. Sci. Food. Agric. 80: 877-900.
Ishiguro, K. 1994. Using simulation models to explore better strategies for the management of blast disease in temperate rice pathosystems. pp. 435-449. In Rice Blast Disease.
Johnson, C. W., Carnahan, H. L., Tseng, S. T., Oster, J. J., and Hill, J. E. 1986. Registration of ‘M-202’ rice. Crop Science 26:198. Jolly et al. 1993. Theor. Appl. Genet. 86:589-597.
Jolly, C. J., S. Rahman, A. A. Kortt, and T. J. V. Higgins. 1993. Characterization of the wheat Mr 15000 “grain-softness protein” and analysis of the relationship between its accumulation in the whole seed and grain softness. Theor. Appl. Genet. 86:589-597.
Kagan, B. L., Selsted, M. E., Ganz, T., and Lehrer, R. I. 1990. Antimicrobial defensin peptides form voltage-dependent ion-permeable channels in planar lipid bilayer membranes. Proc. Natl. Acad. Sci. USA 87:210-214.
Kauffman, H. E., Ebron, T. and Merca, S. 1977. Rice disease survey: 1975. Int'l. Rice Res. Newsl. 2:8-9.
Kim, C. K. 1994. Blast management in high input, high yield potential, temperature rice ecosystems. Pages 451-463, In: Rice Blast Disease.
Kooijman, M., Orsel, R., Hessing, M., Hamer, R. J., and Bekkers, A. C. A. P. A. 1997. Spectroscopic characterization of the lipid-binding properties of wheat puroindolines. J. Cereal Sci. 26:145-159.
Konopka, I., D. Rotkiewicz, and M. Tanska. 2004. Wheat endosperm hardness. Part II. Relationships to content and composition of flour lipids. Eur. Food. Res. Technol. 220:20-24.
Krishnamurthy, K., and M. J. Giroux. 2001. Expression of wheat puroindoline genes in transgenic rice confers grain softness. Nature Biotechnology 19:162-166.
Law, C. N., C. F. Young, J. W. S. Brown, J. W. Snape, and J. W. Worland. 1978. The study of grain protein control in wheat using whole chromosome substitution lines. pp. 483-502. In Seed protein improvement by nuclear techniques. International Atomic Energy Agency, Vienna, Austria.
Lee, F. N. 1994. Rice breeding programs, blast epidemics and blast management in the United States. pp. 489-500. In Rice Blast Disease.
Lin, W., Anuratha, C. S., Datta, K., Potrykus, I., Muthukrishnan, S, and Datta, S. K. 1995. Genetic engineering of rice for resistance to sheath blight. Bio/Technology, 13:686-691.
Logemann, J., Jach, G., Tommerup, H., Mundy, J. and Schell, J. 1992. Expression of a barley ribosome-inactivating protein leads to increased fungal protection in transgenic tobacco plants. Bio/Technology 10:305-308.
Lanning, S. P., Talbert, L. E., McNeal, F. H., Alexander, W. L., McGuire, C. F., Bowman, H., Carlson, G., Jackson, G., Eckhoff, J., and Kushnak, G. 1992. Registration of ‘Hi-Line’ wheat. Crop Sci. 32: 283-284.
Letang, C., Samson, M. F., Lasserre, T. M., Chaurand, M., and Abecassis, J. 2002. Production of starch with very low protein content from soft and hard wheat flours by jet milling and air classification. Cereal Chem. 79:535-543.
Lillemo, M., and Morris C. F. 2000. A leucine to proline mutation in puroindoline b is frequently present in hard wheats from Northern Europe. Theor. Appl. Genet. 100:1100-1107.
LMC International Ltd., 2002. Evaluation of the community policy for starch and starch products. Published online at http://www.ienica.net usefulreports/starch.pdf.
Lanning, S. P., L. E. Talbert, C. F. McGuire, H. F. Bowman, G. D. Carlson, J. L. Jackson, G. D. Eckhoff, R. N. Kushnak, R. N. Stougaard, and G. F. Stallknecht. 1995. Registration of ‘McNeal’ wheat. Crop Sci. 34:1126-1127.
Lillemo, M., and C. F. Morris. 2000. A leucine to proline mutation in puroindoline b is frequently present in hard wheats from Northern Europe. Theor. Appl. Genet. 100:1100-1107.
Marion, D., Gautier, M.-F., Joudrier, P., Ptak, M., Pezolet, M., Forest, E., Clark, D. C., and Broekaert, W. 1994. Structure and function of wheat lipid binding proteins. pp. 175-180. In Wheat Kernel Proteins-Molecular and Functional Aspects. Universita degli studi della tuscia. S Martino al Cimino, Sep. 28-30, 1994. Viterbo, Italy.
Martinez-Espinoza, A. D., S. A. Gerhardt, and J. E. Sherwood. 1993. Morphological and mutational analysis of Ustilago hordei mating. Exp. Mycol. 17:200-214.
Mattern, P. J., R. Morris, J. W. Schmidt, and V. A. Johnson. 1973. Location of genes for kernel properties in the wheat cultivar ‘Cheyenne’ using chromosome substitution lines. pp. 703-707. In E. R. Sears, and L. M. S. Sears (ed.) Proc. Int. Wheat Genet. Symp., 4th, Columbia, Mo. Aug. 1-6, 1973. Agric. Exp. Sta., Univ. Missouri, Columbia, Mo.
Molina, A., Diaz, I., Vasil, I. K., Carbonero, P., and Garcia-Olmedo, F. 1996. Two cold-inducible genes encoding lipid transfer protein LTP4 from barley show differential responses to bacterial pathogens. Mol. Gen. Genet. 252:162-168.
Molina, A., and Garcia-Olmedo, F. 1997. Enhanced tolerance to bacterial pathogens caused by the transgenic expression of barley lipid transfer protein LTP2. The Plant Journal 12:669-675.
Molina, A., Segura, A. and Garcia-Olmedo, F. 1993. Lipid transfer proteins (nsLTPs) from barley and maize leaves are potent inhibitors of bacterial and fungal plant pathogens. FEBS Letters 316:119-122.
Morris et al. 1994. J. Cereal Sci, 21:167-174. National Institute of Agricultural Sciences, Japan. 1954. Insects and diseases of rice plants in Japan. Paper presented at the 4th session, International Rice Commission, Tokyo, 33 p.
Marion, D., Gautier, M. F., Joudrier, P., Ptak, M., Pezolet, M., Forest, E., Clark, D. C. and Broekaert, W. 1994. Structure and function of wheat lipid binding proteins Pages 175-180 in: S.
Martino al Cimino (ed.) Wheat kernel proteins—Molecular and functional aspects: Universita degli studi della tuscia. Viterbo, Italy.
Martin, J. M., Frohberg, R. C., Morris, C. F., L. E. Talbert, L. E, and Giroux, M. J. 2001. Milling and bread baking traits associated with puroindoline sequence type in hard red spring wheat. Crop Sci. 41:228-234.
Martin, J. M. Meyer, F. D. Morris, C. F. and Giroux, M. J. 2007. Pilot scale milling characteristics of transgenic isolines of a hard wheat over-expressing puroindolines. Crop Sci. 47:497-504.
Martin, J. M., Meyer, F. D., Smidansky, E. D., Wanjugi, H., Blechl, A. E., and Giroux M. J. 2006. Complementation of the Pina (null) allele with the wild type Pina sequence restores a soft phenotype in transgenic wheat. Theor. Appl. Genet. 113:1563-1570.
Morris, C. F. and Allan, R. E. 2001. Registration of hard and soft near-isogenic lines of hexaploid wheat genetic stocks. Crop Sci. 41:935-936.
Morris, C. F., Greenblatt, G. A., Bettge, A. D., and Malkawi, H. I. 1994. Isolation and characterization of multiple forms of friabilin. J. Cereal Sci. 21:167-174.
Morris, C. F., King, G. E., Allan, R. E., and Simeone, M. C. 2001a. Identification and characterization of near-isogenic hard and soft hexaploid wheats. Crop Sci. 41:211-217.
Morris, C. F., Lillemo, M., Simeone, M. C., Giroux, M. J., Babb, S. L., and Kidwell, K. K. 2001b. Prevalence of puroindoline grain hardness genotypes among historically significant North American spring and winter wheats. Crop Sci. 41:218-228.
Morris, C. F., and Rose, S. P. 1996. Wheat. Pages 3-54 in: R. J. Henry and P. S. Kettlewell (ed.) Cereal grain quality, Chapman and Hall, London.
Morrison, W. R., Law, C. N., Wylie, L. J., Conventry, A. M. and Seeking, J. 1989. The effect of group 5 chromosomes on the free polar lipids and breadmaking quality of wheat. J. Cereal Sci. 9:41-52.
Martin, J. M., F. D. Meyer, E. D. Smidansky, H. Wanjugi, A. E. Blechl, and M. J. Giroux (2006) Complementation of the Pina (null) allele with the wild type Pina sequence restores a soft phenotype in transgenic wheat. Theor Appl Genet 113:1563-1570.
Morrison, W. R. 1977. Cereal lipids. Proc. Nutr. Soc. 1977, 36, 143-148.
Morrison, W. R., S. L. Tan, and K. D. Hargin. 1980. Methods for the quantitative analysis of lipids in cereal grains and similar tissues.
Oda. S., and J. D. Schofield. 1997. Characterization of friabilin polypeptides. Cereal Sci. 26(1), 29-36.
Ohm, J. B., and O. K. Chung. 1999. Estimation of free glycolipids in wheat flour by HPLC. Cereal Chem. 76:873-876.
Ohm, J. B., and O. K. Chung. 2002. Relationships of free lipids with quality factors in hard winter wheat flours. Cereal Chem. 79(2):274-278.
Ou, S. H.1985. Rice Diseases. 2nd edition Commonwealth Mycological Institute, Kew, U.K. 380p.
Padmanaban, S. Y. 1965. Estimating losses from rice blast in India. pp. 203-221. In Rice Blast Disease, John Hopkins Press, Baltimore, Md.
Parris, N., Moreau, R. A., Johnston, D. B., Singh, V., and Dickey, L. C. 2006. Protein distribution in commercial wet- and dry-milled corn germ. J. Agric. Food Chem. 54:4868-4872.
Pomeranz, Y, and P. C. Williams. 1990. Heat grain hardness: its genetic, structural and biochemical background, measurements and significance. In: Pomeranz Y. (ed) Advances in Cereal Science and Technology, vol. 10. American Association of Cereal Chemistry, St Paul, Minn., pp 471-544.
Pan, Z., W. Song, F. Meng, L. Xu, B. Liu, and J. Zhu. 2004. Characterization of genes encoding wheat grain hardness in Chinese cultivar GaoCheng 8901. Cereal Chem. 81:287-289.
Riede, C. R., and J. A. Anderson. 1996. Linkage of RFLP markers to an aluminum tolerance gene in wheat. Crop Sci. 36:905-909.
Rogers S. G., Klee H. J., Horsch R. B., Farley R. T. 1987. Improved vectors for plant transformation: Expression cassette vectors and new selectable markers. Methods in Enzymol. 153:253-277.
Roy, A. K. 1993. Sheath blight of rice in India. Indian Phytopathology 46:197-205. Schiffer, M., Chang, C. H., and Stevens, F. J. 1992. The functions of tryptophan residues in membrane proteins. Prot. Engin. 5:213-214.
SAS Institute, Inc. 2004. SAS/STAT 9.1 User's guide. SAS Institute Inc., Cary, N.C.
Shen, M., and Lin, J. Y. 1994. The economic impact of rice blast disease in China. pp. 321-331. In Rice Blast Disease. R. S. Zeiglar, S. Leong, and P. S. Teng, eds. CAB International, Wallingford, U.K.
Sivamani, E., Shen, P., Opalka, N., Beachy, R. N., and Fauquet, C. M. 1996. Selection of large quantities of embryogenic calli from indica rice seeds for production of fertile transgenic plants using the biolistic method. Plant Cell Reports 15:322-327.
Smith, H. B. 1999. More than just a surface thing: Rice infection by Magnaporthe grisea. Plant Cell 11:1815-1817.
Song, W.-Y., Wang, G.-L., Chen, L.-L., Kim, H.-S., Pi, L.-Y., Holsten, T., Gardner, J., Wang, B., Zhai, W.-X., Zhu, L.-H., Fauquet, C. and Ronald, P. 1995. A receptor kinase-like protein encoded by the rice disease resistance gene, xa21. Science 270:1804-1806.
Symes, K. J. 1965. The inheritance of grain hardness in wheat as measured by the particle size index. Aust. J. Agric. Res. 16:113-123.
Sayaslan, A. 2006. Characterization of volatile compounds of starches extracted with aqueous solutions. Int. J. Food Properties. 9:463-473.
Sayaslan, A., Seib, P. A., and Chung, O. K. 2006. Wet-milling properties of waxy wheat flours by two laboratory methods. Journal of Food Engineering. J. Food Engineer. 72:167-178.
Swan, C. G, Bowman, J. G. P., Martin, J. M., and Giroux, M. J. 2006a. Increased puroindoline levels slow ruminal digestion of wheat (Triticum aestivum L.) starch by cattle. J. Anim. Sci. 84:641-650.
Symes, K. J. 1965. The inheritance of grain hardness in wheat as measured by particle size index. Aust. J. Agric. Res. 16:113-123.
Simmonds, D. H. 1974. Chemical basis of hardness and vitreosity in wheat kernel. Baker's Digest 48(5) 16-29.
Sourdille, P., M. R. Perretant, G. Channet, P. Leroy, M. F. Gautier, P. Joudrier, J. C. Nelson, M. E. Sorrells, and M. Bernard. 1996. Linkage between RFLP markers and genes affecting kernel hardness in wheat. Theor. Appl. Genet. 93, 580-586.
Swan, C. G., F. D. Meyer, A. C. Hogg, J. M. Martin, and M. J. Giroux. 2006. Puroindoline b limits binding of puroindoline a to starch and grain softness. Crop Sci. 46(4): 1656-1665.
Symes, K. J. 1969. Influence of a gene causing hardness on the milling and baking quality of two wheats. Aust. J. Agric. Res, 20:971-979.
Tester, R. F., Patel, T., and Harding, S. E. 2006. Damaged starch characterisation by ultracentrifugation. CARBOHYDRATE RESEARCH. 341:130-137.
Tsai, W. H.1970. Studies on the relation between rice blast disease. 1. Observations on the host range of rice sheath blight fungus, Pellicularia sasakii on weeds. J. Taiwan Agric. Res. 19:48-50.
Terras, F. R. G., Schoofs, H. M. E., Thevissen, K., Osborn, R. W., Vanderleyden, J., Cammue, B. P. A., and Broekaert, W. F. 1993. Synergistic enhancement of the antifungal activity of wheat and barley thionins by radish and oilseed rape 2S albumins and by barley trypsin inhibitors. Plant Physiol. 103:1311-1319.
Tranquilli, G., D. Lijavetzky., G. Muzzi, and J. Dubcovsky. 1999. Genetic and physical characterization of grain texture-related loci in diploid wheat. Mol. Gen. Genet. 262:846-850.
United States Department of Agriculture. Mar. 9, 2007. World Agricultural Supply and Demand Estimates-444. ISSN:1554-9089. Published on line at http://usda.mannlib.cornell.edu/.
Vasil, V., Srivastava, V., Castillo, A. M., Fromm, M. E., and Vasil, I. K. 1993. Rapid production of transgenic wheat plants by direct bombardment of cultured immature embryos. Bio/Technology 11:1553-1558.
Van Der Borght, A., Goesaert, H., Veraverbeke, W. S, and Delcour, J. A. 2005. Fractionation of wheat and wheat flour into starch and gluten: overview of the main processes and the factors involved. J. Cereal Sci. 41:221-237
Vignaux, N., Fox, S. R., and Johnson, L. A. 2006. A 10-g laboratory wet-milling procedure for maize and comparison with larger scale laboratory procedures. Cereal Chem. 83:482-490.
Weeks, J. T., Anderson, O. D., and Blechl, A. E. 1993. Rapid production of multiple independent lines of fertile transgenic wheat (Triticum aestivum). Plant Physiol. 102:1077-1084.
Willets, D. A and Sherwood, J. E. 1999. Polymerase chain reaction detection of Ustilago hordei in leaves of susceptible and resistant barley varieties. Phytopathology 89:212-217.
Wanjugi, H. W., A. C. Hogg, J. M. Martin, and M. J. Giroux. 2007a. The role of puroindoline a and b individually and in combination on grain hardness and starch association. Crop Sci 47 (1):67-76.
Wanjugi, H. W., J. M. Martin, and M. J. Giroux. 2007b. The influence of puroindoline a and b individually and in combination upon wheat milling and bread baking traits. Crop Sci. Submitted January 2007.
Wolf, M. J. 1964. Wheat starch. Pages 6-9 in: Methods in Carbohydrate Chemistry, Vol 4. R. L. Whistler, ed. Academic press: New York.
Zehr, B. E., Eckhoff, S. R., Nyquist, W. E., and Keeling, P. L. 1996. Heritability of product fractions from wet milling and related properties of maize grain. Crop Sci. 36:1159-1165.
Zeigler, R. S., S. Leong, and P. S. Teng, eds. CAB International, Wallingford, U.K. Kozaka, T. 1965. Ecology of Pellicularia sheath blight of rice plant and its chemical control. Ann Phytopathol. Soc. Japan. 31:179-185.
R. S. Zeigler, S. Leong, and P. S. Teng, eds. CAB International, Wallingford, U.K. Fromm, M. E., Morrish, F. Armstrong, C. Williams, R. Thomas, J., and Klein, T. M. 1990. Inheritance and expression of chimeric genes in the progeny of transgenic maize plants. Bio/Technology 8:833-839.
R. S. Zeigler, S. Leong, and P. S. Teng, eds. CAB International, Wallingford, U.K. Jameson, B. A., and Wolf, H.1988. The antigenic index: a novel algorithm for predicting antigenic determinants. CABIOS 4:181-186.
R. S. Zeigler, S. Leong, and P. S. Teng, eds. CAB International, Wallingford, U.K. Lee, F. N. and Rush, M. C. 1983. Rice sheath blight: a rice disease. Plant Disease 67:829-832.

We claim:

1. A method of extracting starch from a grain comprising separating starch granules and proteins in the grain, wherein the grain is obtained from a plant expressing an introduced nucleic acid coding for a puroindoline.

2. The method of claim 1 wherein the plant is selected from the Genera consisting of Triticum, Sorghum, Oryza, Hordeum, Zea, Secale, Triticale and Avena.

3. The method of claim 1 wherein the puroindoline is selected from the group consisting of puroindoline A, puroindoline B, and both puroindoline A and B.

4. The method of claim 1 wherein the starch granules and the protein are separated by wet milling.

5. The method of claim 1 wherein the plant is the progeny of a plant transformed with the introduced nucleic acid and the progeny inherits and expresses said nucleic acid.


SEQUENCE LISTING (13 sequences)

SEQ ID NO: 1 - DNA, 631 bp, Triticum aestivum
  CDS (43)..(489)
ctcatctattcatctccacctgcaccaaaacacactgacaacatgaaggccctcttcctcataggactgcttgctctggtagcgagcaccgcctttgcgcaatatagcgaagttgttggcagttacgatgttgctggcgggggtggtgctcaacaatgccctgtagagacaaagctaaattcatgcaggaattacctgctagatcgatgctcaacgatgaaggatttcccggtcacctggcgttggtggaaatggtggaagggaggttgtcaagagctccttggggagtgttgcagtcggctcggccaaatgccaccgcaatgccgctgcaacatcatccaggggtcaatccaaggcgatctcggtggcatcttcggatttcagcgtgatcgggcaagcaaagtgatacaagaagccaagaacctgccgcccaggtgcaaccagggccctccctgcaacatccccggcactattggctattactggtgatgtagcttccatttatgactagctaataaactgtcacataccactgcgtgtgacaaataaaagtggtcatggaataatttatgaataaaatttcagcatgtgcctgcgcgaggtgtctatagcaaaaaaaaaaaaaaaaaaa

SEQ ID NO: 2 - protein, 148 aa, Triticum aestivum
MKALFLIGLLALVASTAFAQYSEVVGSYDVAGGGGAQQCPVETKLNSCRNYLLDRCSTMKDFPVTWRWWKWWKGGCQELLGECCSRLGQMPPQCRCNIIQGSIQGDLGGIFGFQRDRASKVIQEAKNLPPRCNQGPPCNIPGTIGYYW

SEQ ID NO: 3 - DNA, 598 bp, Triticum aestivum
  CDS (17)..(463)
aaacaacattgaaaacatgaagaccttattcctcctagctctccttgctcttgtagcgagcacaaccttcgcgcaatactcagaagttggcggctggtacaatgaagttggcggaggaggtggttctcaacaatgtccgcaggagcggccgaagctaagctcttgcaaggattacgtgatggagcgatgtttcacaatgaaggattttccagtcacctggcccacaaaatggtggaagggcggctgtgagcatgaggttcgggagaagtgctgcaagcagctgagccagatagcaccacaatgtcgctgtgattctatccggcgagtgatccaaggcaggctcggtggcttcttgggcatttggcgaggtgaggtattcaaacaacttcagagggcccagagcctcccctcaaagtgcaacatgggcgccgactgcaagttccctagtggctattactggtgatgatatagcctctattcgtgccaataaaatgtcacatatcatagcaagtggcaaataagagtgctgagtgatgatctatgaataaaatcacccttgtatattgatctgtgttcgagaaaaaaaaaaaaaaaaaaa

SEQ ID NO: 4 - protein, 148 aa, Triticum aestivum
MKTLFLLALLALVASTTFAQYSEVGGWYNEVGGGGGSQQCPQERPKLSSCKDYVMERCFTMKDFPVTWPTKWWKGGCEHEVREKCCKQLSQIAPQCRCDSIRRVIQGRLGGFLGIWRGEVFKQLQRAQSLPSKCNMGADCKFPSGYYW

SEQ ID NO: 5 - DNA, 598 bp, Triticum aestivum
  CDS (17)..(463)
aaacaacattgaaaacatgaagaccttattcctcctagctctccttgctcttgtagcgagcacaaccttcgcgcaatactcagaagttggcggctggtacaatgaagttggcggaggaggtggttctcaacaatgtccgcaggagcggccgaagctaagctcttgcaaggattacgtgatggagcgatgtttcacaatgaaggattttccagtcacctggcccacaaaatggtggaagagcggctgtgagcatgaggttcgggagaagtgctgcaagcagctgagccagatagcaccacaatgtcgctgtgattctatccggcgagtgatccaaggcaggctcggtggcttcttgggcatttggcgaggtgaggtattcaaacaacttcagagggcccagagcctcccctcaaagtgcaacatgggcgccgactgcaagttccctagtggctattactggtgatgatatagcctctattcgtgccaataaaatgtcacatatcatagcaagtggcaaataagagtgctgagtgatgatctatgaataaaatcacccttgtatattgatctgtgttcgagaaaaaaaaaaaaaaaaaaa

SEQ ID NO: 6 - protein, 148 aa, Triticum aestivum
MKTLFLLALLALVASTTFAQYSEVGGWYNEVGGGGGSQQCPQERPKLSSCKDYVMERCFTMKDFPVTWPTKWWKSGCEHEVREKCCKQLSQIAPQCRCDSIRRVIQGRLGGFLGIWRGEVFKQLQRAQSLPSKCNMGADCKFPSGYYW

SEQ ID NO: 7 - DNA, 35 bp, Artificial Sequence
  PA5BH primer used for amplifying pinA coding sequences using the RT-PCR
cgggatccaacaatgaaggccctcttcctcatagg

SEQ ID NO: 8 - DNA, 35 bp, Artificial Sequence
  PA3BH primer used for amplifying pinA coding sequences using the RT-PCR technique
ggatcccgccagtaatagccaatagtgccggggat

SEQ ID NO: 9 - DNA, 35 bp, Artificial Sequence
  PB5BH primer used for amplifying pinB coding sequences using the RT-PCR technique
cgggatccaacaatgaagaccttattcctcctagc

SEQ ID NO: 10 - DNA, 32 bp, Artificial Sequence
  PA3BH primer used for amplifying pinB coding sequences using the RT-PCR technique
ggatcccgccagtaatagccactagggaactt

SEQ ID NO: 11 - DNA, 24 bp, Triticum aestivum
ccaccagtaatagccactagggaa

SEQ ID NO: 12 - DNA, 20 bp, Triticum aestivum
gtagtgagcactactattgc

SEQ ID NO: 13 - DNA, 19 bp, Triticum aestivum
gagccttccctccaagtgc